SEQUENCE LISTING (3 sequences)

SEQ ID NO: 1 - DNA, 28 bp, synthetic construct
  source 1..28: mol_type = other DNA
  misc_binding 1: bound_moiety = dibenzylcyclooctyne (DBCO) molecule coupled to 5' end
cctatcccctgtgtgccttggcagtctc

SEQ ID NO: 2 - DNA, 28 bp, synthetic construct
  misc_feature 1..28: note = Synthetic sequence
  source 1..28: mol_type = other DNA
gagactgccaaggcacacaggggatagg

SEQ ID NO: 3 - DNA, 48 bp, synthetic construct
  stem_loop 1..48: note = hairpin molecule
  misc_feature 1: note = dibenzylcyclooctyne (DBCO) molecule linked to 5' end
  modified_base 29: mod_base = OTHER; note = DNA uracil
  misc_feature 48: note = dideoxycytosine
  source 1..48: mol_type = other DNA
cctatcccctgtgtgccttggcagtctcttttttttgactgccaaggc

(19) United States
(12) Patent Application Publication
(10) Pub. No.: US 2024/0117411 A1
(43) Pub. Date: Apr. 11, 2024

(54) METHODS FOR PREPARING CAPTURE SUBSTRATES

(71) Applicant: Ultima Genomics, Inc., Fremont, CA (US)

(72) Inventors: Robert ONO, Palo Alto, CA (US); Daniel MAZUR, San Diego, CA (US); Florian OBERSTRASS, Menlo Park, CA (US)

(21) Appl. No.: 18/540,604

(22) Filed: Dec. 14, 2023

Related U.S. Application Data
(63) Continuation of application No. PCT/US2022/033468, filed on Jun. 14, 2022.
(60) Provisional application No. 63/210,527, filed on Jun. 15, 2021.

(51) Int. Cl. C12Q 1/6806

(52) U.S. Cl. CPC C12Q 1/6806

(57) ABSTRACT

Methods of controlling the density or number of nucleic acid molecules coupled to solid supports are provided. Capture substrates produced thereby or comprising a predetermined density or number of nucleic acids coupled thereto are also provided.

### CROSS-REFERENCE

This application is a Continuation of International Application No. PCT/US2022/033468, filed Jun. 14, 2022, which claims the benefit of U.S. Provisional Patent Application No. 63/210,527, filed Jun. 15, 2021, which are entirely incorporated by reference herein.

### REFERENCE TO AN ELECTRONIC SEQUENCE LISTING

The contents of the electronic sequence listing (406C1_SeqListing.xml; Size: 6,208 bytes; and Date of Creation: Dec. 8, 2023) is herein incorporated by reference in its entirety.

### BACKGROUND

The detection, quantification and sequencing of cells and biological molecules is important for molecular biology and medical applications, such as diagnostics. Genetic testing may be useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information.

Nucleic acid sequencing is a process that can be used to provide sequence information for a nucleic acid sample. Such sequence information may be helpful in diagnosing and/or treating a subject with a condition. For example, the nucleic acid sequence of a subject may be used to identify, diagnose, and potentially develop treatments for genetic diseases. As another example, research into pathogens may lead to treatment of contagious diseases.

Nucleic acid sequencing may comprise sequencing of nucleic acid molecules coupled to particles such as beads. However, the use of such systems may reduce sequencing quality, for example, by aggregation of particles through associations between nucleic acid molecules bound to the particles.

For the amplification of a pool of different polynucleotides having unknown or partially unknown sequences, the preparation of a polynucleotide library generally takes place. The library molecules may each contain a universal sequence that can be used to capture the molecules to capture substrates. A single nucleic acid molecule is generally captured to each substrate and clonal amplification is carried out on the substrate to produce a colony that can be sequenced. To facilitate capture of only properly produced library molecules, the substrate may contain nucleic acid primers that are reverse complementary to the common sequence on the library molecules.

Conjugation of primers to a solid substrate can be performed in a myriad of ways, but the process is relatively expensive due to the cost of synthesizing millions of primers to conjugate to thousands of substrates. Additionally, the presence of single-stranded nucleic acids on the surface of the substrates can cause clumping and loss of useful substrate.

### SUMMARY

Despite the prevalence of biological sample processing systems and methods, such systems and methods may have low efficiency that can be time-intensive and wasteful of valuable resources, such as reagents. The processing of a biological sample may include generating a capture substrate, for example, by the binding of single-stranded primers to supports. Recognized herein is a need for improved methods of generating capture substrates and their preparation in advance of library molecule capture and amplification.

The present invention provides methods of controlling the density or number of nucleic acid molecules coupled to solid supports. Capture substrates produced thereby or comprising a predetermined density or number of nucleic acids coupled thereto are also provided.

According to a first aspect, there is provided a method of controlling density of nucleic acid molecules coupled to solid supports. The method comprises (a) providing a plurality of solid supports, where each solid support comprises a plurality of first coupling moieties. The method continues by (b) contacting the plurality of solid supports with a plurality of nucleic acid molecules under conditions sufficient to couple the first coupling moiety to a second coupling moiety of the plurality of nucleic acid molecules to produce a coupled solid support. Each nucleic acid molecule comprises either: (i) a first strand and a second strand, or (ii) a third strand comprising a double-stranded region in which a first region of the third strand is hybridized to a second region of the third strand. The first strand comprises a 5′ linked second coupling moiety configured to couple to the first coupling moiety. The third strand comprises a 5′ linked second coupling moiety configured to couple to the first coupling moiety. The method proceeds, thereby producing a population of coupled solid supports that are coupled to nucleic acid molecules, wherein each coupled solid support is coupled to the nucleic acid molecules at a density that is less than a predetermined percentage of a maximal density.

According to some embodiments, the maximal density comprises all of the plurality of first coupling moieties of the solid support being coupled to a nucleic acid molecule.

According to some embodiments, the predetermined percentage is about 50%, about 60%, about 70%, about 80%, or about 90%.

According to some embodiments, the density that each coupled solid support is coupled to nucleic acid molecules is less than about 75,000 nucleic acid molecules per cubic micron. According to certain embodiments, the density that each coupled solid support is coupled to nucleic acid molecules is less than about 20,000 nucleic acid molecules per square micron.

According to another aspect, there is provided a method of controlling a number of nucleic acid molecules coupled to solid supports. The method comprises (a) providing a plurality of solid supports, where each solid support comprises a plurality of first coupling moieties. The method continues by (b) contacting the plurality of solid supports with a plurality of nucleic acid molecules under conditions sufficient to couple the first coupling moiety to a second coupling moiety of the plurality of nucleic acid molecules to produce a coupled solid support. Each nucleic acid molecule comprises either: (i) a first strand and a second strand, or (ii) a third strand comprising a double-stranded region in which a first region of the third strand is hybridized to a second region of the third strand. The first strand comprises a 5′ linked second coupling moiety configured to couple to the first coupling moiety. The third strand comprises a 5′ linked second coupling moiety configured to couple to the first coupling moiety. The method proceeds, thereby producing a population of coupled solid supports that are coupled to nucleic acid molecules, wherein each coupled solid support is coupled to less than a threshold number of nucleic acid molecules.

According to some embodiments, the threshold number of nucleic acid molecules is about 75,000 nucleic acid molecules. According to some embodiments, the threshold number of nucleic acid molecules is about 10,000 nucleic acid molecules. According to some embodiments, the threshold number of nucleic acid molecules is less than a maximum number of nucleic acid molecules that can be coupled to the solid support.

According to some embodiments, each nucleic acid molecule comprises (1) the first strand and the second strand, and (2) a double-stranded region in which at least a portion of the first strand is hybridized to at least a portion of the second strand.

According to some embodiments, the first coupling moiety does not comprise a nucleic acid.

According to some embodiments, the method further comprises (c) denaturing the second strand from the first strand to produce a coupled solid support that is coupled to a plurality of single-stranded primers.

According to some embodiments, the solid support is a bead. According to some embodiments, the bead is a microbead. According to some embodiments, the microbead has a diameter of between about 0.2 and about 2.5 microns (μm).

According to some embodiments, the first coupling moiety and the second coupling moiety couple by forming a covalent bond. According to some embodiments, the first coupling moiety and the second coupling moiety couple by click chemistry.

According to some embodiments, the first coupling moiety is an azide molecule and the second coupling moiety is a diarylcyclooctyne moiety, or the first coupling moiety is a diarylcyclooctyne moiety and the second coupling moiety is an azide molecule. According to some embodiments, the diarylcyclooctyne moiety is dibenzylcyclooctyne (DBCO).

According to some embodiments, the first strand comprises 15 to 45 nucleotides. According to some embodiments, the second strand comprises 15 to 650 nucleotides.

According to some embodiments, the method further comprises contacting the coupled solid support with a 5′ to 3′ nuclease capable of degrading a 5′ unhybridized region of the second strand under conditions sufficient to degrade the 5′ unhybridized region of the second strand and leave a hybridized region of the second strand hybridized to the first strand.

According to some embodiments, the second strand of the nucleic acid molecules is linked at a 5′ terminus to a non-nucleotide polymer. According to some embodiments, the second strand is linked to the non-nucleotide polymer by a covalent bond. According to some embodiments, the second strand is linked to the non-nucleotide polymer by click chemistry.

According to some embodiments, the non-nucleotide polymer is a synthetic polymer. According to some embodiments, the non-nucleotide polymer has a size of between about 10,000 g/mol to about 700,000 g/mol. According to some embodiments, the non-nucleotide polymer has a radius of between about 1 nm and about 100 nm. According to some embodiments, the non-nucleotide polymer is selected from the group consisting of: a polyethylene glycol (PEG) polymer, a poly-L-glutamate (poly(L-glu)) polymer, and a combination thereof.

According to some embodiments, the method further comprises, after denaturing the second strand from the first strand, collecting the second strands after the denaturing, and reusing them in a method of the invention.

According to some embodiments, the first region of the third strand is a 5′ region of the third strand and the second region of the third strand is a 3′ region of the third strand.

According to some embodiments, the third strand further comprises an unhybridized region between the first region and the second region and wherein the unhybridized region comprises at least one cleavable or excisable base.

According to some embodiments, the method further comprises subjecting the coupled solid support to conditions sufficient to cleave or excise the at least one cleavable or excisable base to produce two separate strands. According to some embodiments, the conditions comprise contacting the nucleic acid molecule with a cleaving agent configured to cleave or excise the at least one cleavable or excisable base.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and any combination thereof.

According to some embodiments, the at least one cleavable or excisable base is selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, a photocleavable base, and any combination thereof. According to some embodiments, the at least one cleavable or excisable base is adjacent to a 3′ end of the first region.

According to some embodiments, the unhybridized region comprises a polyT stretch of at least 5 bases. According to some embodiments, the unhybridized region comprises a plurality of cleavable or excisable bases and wherein cleavage or excision of the plurality of cleavable or excisable bases removes at least a portion of the unhybridized region. According to some embodiments, the unhybridized region comprises a plurality of cleavable or excisable bases and wherein cleavage or excision of the plurality of cleavable or excisable bases removes the unhybridized region.

According to some embodiments, a first cleavable or excisable bases is adjacent to a 3′ end of the first region and wherein a second cleavable or excisable bases is adjacent to 5′ end of the second region.

According to some embodiments, each coupled solid support is coupled to nucleic acid molecules at a density that is less than a predetermined percentage of a maximal density. According to some embodiments, the predetermined percentage is about 50%, about 60%, about 70%, about 80%, or about 90%. According to some embodiments, the maximal density comprises all first coupling moieties of the coupled solid support coupled to a nucleic acid molecule.

According to some embodiments, the density that each coupled solid support is coupled to nucleic acid molecules is less than about 75,000 nucleic acid molecules per cubic micron. According to some embodiments, the density that each solid support is coupled to nucleic acid molecules is less than about 20,000 nucleic acid molecules per cubic micron.

According to some embodiments, less than 50% of the maximal density comprises at least half of all first coupling moieties not coupled to a nucleic acid molecule.

According to some embodiments, the coupled solid support comprises a first portion of the first coupling moieties coupled to the second coupling moiety and a second portion of the first coupling moieties not coupled to the second coupling moiety and wherein an average distance between adjacent first coupling moieties coupled to the second coupling moiety is at least 2 nm.

According to some embodiments, the method further comprises (d) contacting the coupled solid supports with a target nucleic acid molecule, wherein the target nucleic acid molecule comprises a 3′ region of reverse complementarity to the single-stranded primers, under conditions sufficient to hybridize the target nucleic acid molecule to the single-stranded primer to produce a solid support coupled to the target nucleic acid molecule.

According to some embodiments, the method further comprises (e) extending the single-stranded primer from a 3′ end to produce a nucleic acid strand coupled to the solid support and reverse complementary to the target nucleic acid molecule.

According to some embodiments, the method further comprises: clonally amplifying the target nucleic acid molecule using the single-stranded primers coupled to the solid support.

According to some embodiments, the extending, the amplifying, or both comprise contacting the solid support coupled to the target nucleic acid molecule with a polymerase and a plurality of free nucleotides.

According to another aspect, there is provided a capture substrate comprising a plurality of solid supports each coupled to a plurality of nucleic acid primers produced by a method of the invention.

According to another aspect, there is provided a capture substrate, comprising a solid support coupled to a plurality of primers, wherein each primer is hybridized to a complementary nucleic acid molecule and wherein the complementary nucleic acid molecule is devoid of a 5′ unhybridized region of greater than 5 nucleotides.

According to some embodiments, the complementary nucleic acid molecule is devoid of a capture entity.

According to another aspect, there is provided a capture substrate, comprising a solid support comprising first coupling moieties and wherein a first portion of the first coupling moieties are coupled to a nucleic acid molecule and a second portion of the first coupling moieties are not coupled to a nucleic acid molecule and wherein the nucleic acid molecules are present at a density that is less than a predetermined percentage of a maximal density.

According to some embodiments, the predetermined percentage is about 50%, about 60%, about 70%, about 80%, or about 90%.

According to some embodiments, less than about 50% of the maximal density comprises at least half of all first coupling moieties not coupled to a nucleic acid molecule. According to some embodiments, at least 10% of the first coupling moieties are coupled to the nucleic acid molecule.

According to some embodiments, the coupled solid support is coupled to nucleic acid molecules at a density of less than about 75,000 nucleic acid molecules per cubic micron. According to some embodiments, the coupled solid support is coupled to nucleic acid molecules at a density of less than about 10,000 nucleic acid molecules per cubic micron.

According to another aspect, there is provided a capture substrate, comprising a solid support comprising between about 5,000 and about 75,000 nucleic acid molecules coupled thereto, wherein the nucleic acid molecules are between about 15 and about 45 nucleotides in length.

According to some embodiments, the solid support comprises between about 5,000 and about 10,000 nucleic acid molecules coupled thereto. According to some embodiments, the solid support has a diameter of between about 0.2 microns and about 2.5 microns.

According to another aspect, there is provided a capture substrate comprising a solid support comprising a first coupling moiety and an adjacent first coupling moiety, wherein the first coupling moiety and the adjacent first coupling moiety are each coupled to a nucleic acid molecule, and wherein a distance between the first coupling moiety and the adjacent first coupling moiety is at least 2 nm.

According to some embodiments, the solid support comprises a plurality of first coupling moieties not coupled to a nucleic acid molecule and wherein an average distance between all adjacent first coupling moieties is less than 2 nm. According to some embodiments, the average distance between all adjacent first coupling moieties is less than 1 nm.

According to some embodiments, the solid support is a bead. According to some embodiments, the bead is a microbead. According to some embodiments, the bead has a diameter of between about 0.2 and about 2.5 microns.

According to some embodiments, the nucleic acid molecule is a single-stranded primer. According to some embodiments, the single-stranded primer comprises 15 to 45 nucleotides. According to some embodiments, the nucleic acid molecule is a double-stranded nucleic acid molecule and where a strand not coupled to the solid support is devoid of a 5′ single-stranded region of greater than 5 nucleotides.

According to some embodiments, the solid support is coupled to the nucleic acid molecule by a covalent bond. According to some embodiments, the solid support has been coupled to the nucleic acid molecule via a click reaction. According to some embodiments, the solid support is coupled to a 5′ end of the nucleic acid molecule.

According to another aspect, there is provided a composition comprising the capture substrate of the invention. According to some embodiments, the capture substrate is present at a density sufficient for hybridization between single-stranded nucleic acid molecules of different capture substrates and wherein the capture substrate is a capture substrate of the invention or wherein the nucleic acid molecule is a double-stranded nucleic acid molecule.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIGS. 1A-1B illustrate certain methods of the invention that employ a mock-template second strand as a bulky molecule, (FIG. 1A) without and (FIG. 1B) with a nuclease digestion of the mock-template before denaturing.

FIGS. 2A-2C show schematic depictions of the density of nucleic acid molecules produced on supports produced by (FIG. 2A) conventional methods using only single-stranded primers and (FIGS. 2B-2C) certain methods of the invention using (FIG. 2B) a mock-template or non-nucleotide polymer and (FIG. 2C) a hairpin molecule.

FIG. 3 shows a schematic depiction of an example method of the invention that employs a non-nucleotide polymer as a bulky molecule.

FIGS. 4A-4B show schematic depictions of certain methods of the invention that employ as a bulky molecule a hairpin molecule containing (FIG. 4A) one or (FIG. 4B) at least 2 cleavable or excisable bases.

### DETAILED DESCRIPTION

The present invention, in some embodiments, provides methods of controlling the density or amount of nucleic acid molecules coupled to solid supports. Capture substrates produced thereby or comprising a predetermined density or number of nucleic acids coupled thereto are also provided.

The invention is based, at least in part, on the surprising finding that a large number of primers attached to capture substrates, e.g., beads, used to capture library molecules for clonal amplification are wasted and do not end up being used for amplification. When the primers are conjugated to the substrates, they are short single-stranded molecules that can be captured to the huge number of reactive moieties present on the surface of the substrate (e.g., azide groups on beads). In contrast, a library molecule that is captured by a primer is long (e.g., ˜200-650 nucleotides) and its binding to a primer produces a double-stranded molecule with at least twice the diameter of the primer alone. In fact, this long nucleic acid strand does not merely stretch out but rather exists as a random coil in solution which greatly increases the effective diameter of the molecule. As amplification progresses, each newly synthesized strand hence makes use of a single primer but takes up a sufficient amount of space so as to occlude other primers from binding to library molecules.

There is a wide range in the percent primer utilization in cases where a maximal number of primers are conjugated to a given bead. It was found that when short library molecules (e.g., less than ˜200 nucleotides) are used, approximately 50% of the primers are used for clonal amplification. However, as the length of library molecules increases, primer utilization decreases. For example, when longer library molecules (e.g., greater than 400 nucleotides) are used, only ˜15-20% of the primers are actually used in a clonal amplification. Thus, in many cases, ˜80-85% of primers on the substrate go to waste. In the alternative methods provided herein, the density of primers conjugated to the substrate is controlled, and the number of primers attached to beads is decreased by more than 50%. This reduction in primer density surprisingly had no effect on amplification efficiency or the total number of clonal copies produced. The utilization of the primers on the substrate increased to over 30%, and in some cases approached 50%. Thus, the novel methods provided herein produced superior beads, in that they performed capture and clonal amplification equally well, but without the waste of such a large number of primers.

### Definitions

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid may comprise A nucleic acid can comprise a sequence of four natural nucleotide bases: adenine (A); cytosine (C); guanine (G); and thymine (T) (uracil (U) for thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The terms “polynucleotide”, “polynucleotide sequence”, “nucleic acid sequence”, “template nucleic acid,” “target nucleic acid,” “nucleic acid fragment,” “oligonucleotide,” “nucleic acid,” and “nucleic acid molecule” are used interchangeably herein. These terms encompass nucleotide sequences and the like. A polynucleotide may be a polymer of RNA or DNA that is single- or double-stranded, that optionally contains synthetic, non-natural, or altered nucleotide bases. These terms generally refer to polymeric forms of nucleotides of any length, such as deoxyribonucleotides (dNTPs) or ribonucleotides (rNTPs), or analogs thereof.

As used herein, the term “library” refers to a plurality of polynucleotide molecules which share common sequences at their 5′ ends and common sequences at their 3′ ends. In some instances, the sequences and at the 5′ end and the sequences at the 3′ end are different sequences. In some instances, the different sequences are not complementary to each other. In some instances, the polynucleotide molecules are template for subsequent enzymatic reaction. In some instances, the enzymatic reaction is a polymerase reaction. In some instances, the enzymatic reaction is polymerization. In some instances, the enzymatic reaction is amplification.

As used herein, the term “template” refers to the fact that one or both strands of a polynucleotide are capable of acting as templates for template-dependent nucleic acid polymerization. In some instances, a template-dependent nucleic acid polymerization is catalyzed by a polymerase. In some instances, polymerization comprises elongation of a polymer by adjoining moieties, e.g., nucleotides, by formation of phosphor-diester bond(s). In some instances, the polymerization is amplification.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or another derivative thereof. In another example, the analyte can be a protein, and the template nucleic acid is an oligonucleotide that is conjugated to an antibody that binds to the protein, or derivative thereof. Sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence-based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference) Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some instances, “substantial identity” exists over a region of the sequences being compared. In some instances, substantial identity exists over a region of at least 25 residues in length, at least 50 residues in length, at least 100 residues in length, at least 150 residues in length, at least 200 residues in length, or greater than 200 residues in length. In some instances, the sequences being compared are substantially identical over the full length of the sequences being compared. Typically, substantially identical nucleic acid or protein sequences include less than 100% nucleotide or amino acid residue identity as such sequences would generally be considered “identical.”

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or via a non-covalent interaction. A coupling between a first object and a second object may comprise a labile moiety, such as a moiety comprising an ester, vicinal diol, phosphodiester, peptide, glycosidic, sulfone, Diels-Alder, or similar linkage. The strength of a coupling between a first object and a second object may be indicated by a dissociation constant, K_{d}, which indicates the inclination of a coupled object comprising a first object and a second object to dissociate into the uncoupled first and second objects and may be expressed as a ratio of dissociated (e.g., uncoupled) objects to coupled objects.

As used herein, the term “cleavable or excisable base” generally refers to any base or analog of a base (e.g., nucleobase) that can be specifically cleaved and removed or excised from a nucleic acid molecule. The terms “cleavable” and “excisable” as used herein are synonymous and interchangeable. The terms “cleavage” and “excision” as used herein are synonymous and interchangeable. Examples of cleavable bases include, but are not limited to, uracil, 8-oxoguanine (also referred to as 8-hydroxyguanine, 8-oxo-7,8-dihydroguanine, 7,8-dihydro-8-oxoguanine, and 8oxoG herein), inosine, and 2,6-diamino-4-hydroxy-5-formamidopyrimidine (FapyG). In some instances, the uracil is a DNA uracil. In some instances, the uracil is an RNA uracil. Cleavage and/or excision of a cleavable or excisable moiety may be carried out by contacting the cleavable or excisable moiety (e.g., cleavable base) with a cleaving agent. Examples of cleaving agents include, but are not limited to, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), and RNase (e.g., RNaseH, such as RNaseHII). Photocleavable or photoexcisable moieties may be cleaved or excised using appropriate application of energy, such as by contacting the moiety with UV light. In some instances, a cleavable or excisable moiety is a cleavable or excisable base. One or more cleaving agents may be used in combination to cleave or excise a cleavable or excisable moiety. In some instances, the cleavable or excisable base is subjected to condition sufficient to cleave or excise it. In some instances, the suitable conditions comprise contacting the cleavable or excisable base with a cleaving agent configured to cleave or excise the at least one cleavable or excisable base. In an example, the cleavable base may be an RNA base in a DNA backbone, and the cleaving agent may be RNase (e.g., RNaseH or RNaseHII). In such a case, the nucleic acid molecule may not be an RNA molecule. In some instances, the cleavable or excisable base is an RNA base and the nucleic acid molecule s devoid of RNA bases other than the cleavable or excisable base. In another example, the cleavable base may be a uracil DNA base and the cleaving agent may be selected from uracil DNA glycosylase (UDG), apyrimidinic/apurinic endonuclease (APE), Endonuclease VIII and uracil-specific excision reagent (USER) enzyme. For example, the cleaving agent may be UDG. For example, the cleaving agent may be APE. In another example, the cleavable base may be an inosine base and the cleaving agent may be Endonuclease V (Endo V). In another example, the cleavable base may be 2,6-diamino-4-hydroxy-5-formamidopyrimidine (FapyG) base and the cleaving agent may be formamidopyrimidine DNA glycosylase (Fpg). In another example, the cleavable base may be 8-oxo-7,8-dihydroguanine (8oxoG) and the cleaving agent may be 8-oxoguanine glycosylase (OGG1). In another example, the cleavable base may be a photo-cleavable base and the cleaving agent may be light, such as laser light. Application of a cleaving agent may generate a “nick” in a strand of a nucleic acid molecule. Alternatively, or in addition to, another enzyme may be added to generate a nick, or otherwise functionalize a nick. For example, T4 PNK may be added to remove a 3′ phosphate. An enzyme may be used to remove a lesion, such as a 3′ lesion. In some instances, the cleavable or excisable base is an RNA base, and the cleaving agent is RNase H. In some instances, the RNase H is RNase HII. In some instances, the RNA base is a uracil RNA base. In some instances, the cleavable or excisable base is a uracil DNA base, and the cleaving agent is selected from a) UDG, b) UDG and an Endonuclease and c) USER. In some instances, the Endonuclease is Endonuclease VIII.

As used herein, the term “complementary” refers to the ability of polynucleotides to form base pairs (hybridize) with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

The term “hybridization” or “hybridizes” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. RNA sequences can also include complementary G=U or U=G base pairs. Thus, two sequences need not have perfect homology to be “complementary” under the invention. In some instances, complementary comprises at least 70, 75, 80, 85, 90, 95, 97, 99 or 100% homology. Each possibility represents a separate embodiment of the invention. In some instances, complementary comprises at least 70% homology. In some instances, complementary comprises at least 80% homology. In some instances, complementary comprises at least 90% homology. In some instances, complementary comprises 100% homology.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the invention are comprised of nucleotides ranging from 8 to 35 nucleotides. In one embodiment, the primer is at least 8, at least 9, at least 10, at least 11, at least 12, at least 13, at least 14, at least 15, at least 16, at least 17, at least 18, at least 19, at least 20, at least 21 nucleotides, at least 22, at least 23, at least 24, at least 25, at least 26, at least 27, at least 28, at least 29, at least 30, at least 31, at least 32, at least 33, at least 34, or at least 35 nucleotides long, or any value or range therebetween. Each possibility represents a separate embodiment of the invention. In one embodiment, the primer is 10 to 50 nucleotides, 5 to 40 nucleotides, 8 to 45 nucleotides, 20 to 35 nucleotides, 18 to 30 nucleotides, or 20 to 45 nucleotides long. Each possibility represents a separate embodiment of the invention.

As used herein, the term “functional group” refers to a molecule or a moiety within a molecule that can undergo a characteristic molecular reaction when reacted with a specific reactant. Functional groups are well known in the art. Broad categories of functional groups include hydrocarbon functional groups (including alkane, alkene, alkyne and benzene), halogen functional groups (halide, fluoride, chloride, bromide and iodide), oxygen functional groups (hydroxyl, carbonyl, aldehyde, haloformyl, carbonate ester, carboxylate, carboxyl, carboalkyoxy, hydroperoxyl, peroxyl, ether, hemiacetal, hemiketal, acetal, ketal, orthoester, methylendioxy, orthocarbonate ester, carboxylic anhydride), nitrogen function groups (amide, amine, ammonium, imine, imide, azide, diazene, cyanate, isocynate, nitrate, nitrile, isonitrile, nitrite, nitro, oxime, pyridine, carbamate), sulfur functional groups, (thiol, sulfide, disulfide, sulfoxide, sulfone, sulfinic acid, sulfonic acid, sulfonate, thiocyanate, isothiocyanate, thione, thial, thioic O-acid, thioate, dithioic acid, dithioate), phosphorus functional groups (phosphane, phosphonic acid, phosphate) and boron functional groups (boronic acid, boronic acid ester, borinic acid, borinic acid ester).

The term “support” or “substrate,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. An amplification substrate can be placed within or on another substrate, for example, where beads used as amplification substrates are disposed (e.g., immobilized) on a planar surface, or where beads used as amplification substrates are disposed (e.g., immobilized) inside of wells.

The term “bead,” as described herein, generally refers to a solid support, resin, gel (e.g., hydrogel), colloid, or particle of any shape and dimensions. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. For example, a bead may comprise one or more polymers bearing one or more magnetic labels. A magnetic bead may be manipulated (e.g., moved between locations or physically constrained to a given) using electromagnetic forces. A bead may have one or more different dimensions including a diameter. A dimension of the bead (e.g., the diameter of the bead) may be less than about 1 mm, less than about 0.1 mm, less than about 0.01 mm, less than about 0.005 mm, from about 1 nm to about 100 nm, from about 100 nm to about 1 μm, or from about 1 μm to about 10 μm.

### Methods of Controlling Primer Density

By a first aspect, there is provided a method of controlling density of nucleic acid molecules coupled to solid supports, the method comprising: (a) providing a plurality of solid supports, wherein each solid support comprises a plurality of first coupling moieties; and (b) contacting the plurality of solid supports with a plurality of nucleic acid molecules, under conditions sufficient to couple the first coupling moiety to a second coupling moiety of the plurality of nucleic acid molecules to produce a coupled solid support, wherein each nucleic acid molecule comprises a first strand and a second strand, and wherein the first strand comprises the second coupling moiety configured to couple to the first coupling moiety, thereby producing a population of coupled solid supports that is coupled to nucleic acid molecules, wherein each coupled solid support is coupled to the nucleic acid molecules at a density that is less than a maximal density.

By another aspect, there is provided a method of controlling a number of nucleic acid molecules coupled to solid supports, the method comprising: (a) providing a plurality of solid supports, wherein each solid support comprises a plurality of first coupling moieties; and (b) contacting the plurality of solid supports with a plurality of nucleic acid molecules, under conditions sufficient to couple the first coupling moiety to a second coupling moiety of the plurality of nucleic acid molecules to produce a coupled solid support, wherein each nucleic acid molecule comprises a first strand and a second strand, and wherein the first strand comprises the second coupling moiety configured to couple to the first coupling moiety, thereby producing a population of coupled solid supports that is coupled to nucleic acid molecules, wherein each coupled solid support is coupled to fewer than a threshold number of nucleic acid molecules.

In some embodiments, the method produces a capture substrate. In some embodiments, the method produces a solid support. In some embodiments, the solid support is solid for use in the capture of a target nucleic acid molecule. In some embodiments, the solid support is solid for use in a method of clonal amplification. In some embodiments, the clonal amplification is amplification of the target nucleic acid molecule. In some embodiments, the method produces a solid support with a controlled number of nucleic acid molecules conjugated thereto. In some embodiments, the method produces a solid support with a controlled density of nucleic acid molecules conjugated thereto. In some embodiments, the method produces a solid support with a predetermined number of nucleic acid molecules conjugated thereto. In some embodiments, the method produces a solid support with a predetermined density of nucleic acid molecules conjugated thereto. In some embodiments, the method produces a solid support with a number of nucleic acid molecules below a maximum amount conjugated thereto. In some embodiments, the method produces a solid support with a density of nucleic acid molecules below a maximum density conjugated thereto.

In some embodiments, the nucleic acid molecule is DNA, RNA or a mixture of DNA and RNA. In some embodiments, the nucleic acid molecule is DNA. In some instances, the nucleic acid molecule is RNA. In some embodiments, the nucleic acid molecule is cDNA. In some embodiments, the nucleic acid molecule is a locked nucleic acid molecule (LNA). In some embodiments, the nucleic acid molecule is a peptide nucleic acid (PNA). In some embodiments, the nucleic acid molecule is double-stranded. In some instances, the nucleic acid molecule is single-stranded. In some embodiments, the nucleic acid molecule comprises a double-stranded region. In some embodiments, the nucleic acid molecule comprises a single-stranded region.

An oligonucleotide is typically composed of a specific sequence of four nucleotide bases: adenine (A); cytosine (C); guanine (G); and thymine (T) (or uracil (U) instead of thymine (T) if the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

In some embodiments, the nucleic acid molecule comprises at least 5, 6, 7, 8, 9, 10, 11, 12, 13, 14, 15, 20, 25, 30, 35, 40, 45, 50, 60, 70, 80, 90 or 100 nucleotides. Each possibility represents a separate embodiment of the invention. In some embodiments, the nucleic acid molecule comprises at least 10 nucleotides. In some embodiments, the nucleic acid molecule comprises at least 15 nucleotides. In some embodiments, the nucleic acid molecule comprises at most 15, 20, 25, 30, 35, 40, 45, 50, 55, 60, 70, 80, 90, 100, 150, 200, 250, 300, 350, 400, 450, 500, 550, 600, 650, 700, 750, 800, 850, 900, 950 or 1000 nucleotides. Each possibility represents a separate embodiment of the invention. In some embodiments, the nucleic acid molecule comprises at most 650 nucleotides. In some embodiments, the nucleic acid molecule comprises at most 700 nucleotides. In some embodiments, the nucleic acid molecule comprises between 15-800, 15-750, 15-700, 15-650, 15-600, 15-550, 15-500, 20-800, 20-750, 20-700, 20-650, 20-600, 20-550, 20-500, 25-800, 25-750, 25-700, 25-650, 25-600, 25-550, 25-500, 30-800, 30-750, 30-700, 30-650, 30-600, 30-550, 30-500, 35-800, 35-750, 35-700, 35-650, 35-600, 35-550, 35-500, 40-800, 40-750, 40-700, 40-650, 40-600, 40-550, 40-500, 45-800, 45-750, 45-700, 45-650, 45-600, 45-550, or 45-500 nucleotides. Each possibility represents a separate embodiment of the invention. In some embodiments, the nucleic acid molecule comprises between 15 and 650 nucleotides. In some embodiments, the nucleic acid molecule comprises between 15 and 700 nucleotides.

In some instances, the first strand of the nucleic acid molecule comprises at least 5, 6, 7, 8, 9, 10, 11, 12, 13, 14, 15, 16, 17, 18, 19 or 20 nucleotides. Each possibility represents a separate embodiment of the invention. In some instances, the first strand comprises at least 10 nucleotides. In some instances, the first strand comprises at least 15 nucleotides. In some instances, the first strand comprises at most 15, 20, 25, 30, 35, 40, 45, 50, 55, 60, 65, 70, 75, 80, 85, 90, 95, or 100 nucleotides. Each possibility represents a separate embodiment of the invention. In some instances, the first strand comprises at most 45 nucleotides. In some instances, the first strand comprises between 5-100, 10-100, 15-100, 5-90, 10-90, 15-90, 5-80, 10-80, 15-80, 5-70, 10-70, 15-70, 5-60, 10-60, 15-60, 5-55, 10-55, 15-55, 5-50, 10-50, 15-50, 5-45, 10-45, 15-45, 5-40, 10-40, 15-40, 5-35, 10-35 or 15-35 nucleotides. Each possibility represents a separate embodiment of the invention. In some instances, the first strand comprises between 15-45 nucleotides.

In some instances, the second strand of the nucleic acid molecule comprises at least 5, 6, 7, 8, 9, 10, 11, 12, 13, 14, 15, 16, 17, 18, 19 or 20 nucleotides. Each possibility represents a separate embodiment of the invention. In some instances, the second strand comprises at least 10 nucleotides. In some instances, the second strand comprises at least 15 nucleotides. In some instances, the second strand comprises at most 100, 150, 200, 250, 300, 350, 400, 450, 500, 550, 600, 650, 700, 750, 800, 850, 900, 950 or 1000 nucleotides. Each possibility represents a separate embodiment of the invention. In some embodiments, the second strand comprises at most 650 nucleotides. In some embodiments, the second strand comprises at most 700 nucleotides. In some embodiments, the second strand comprises between 15-800, 15-750, 15-700, 15-650, 15-600, 15-550, 15-500, 20-800, 20-750, 20-700, 20-650, 20-600, 20-550, 20-500, 25-800, 25-750, 25-700, 25-650, 25-600, 25-550, 25-500, 30-800, 30-750, 30-700, 30-650, 30-600, 30-550, 30-500, 35-800, 35-750, 35-700, 35-650, 35-600, 35-550, 35-500, 40-800, 40-750, 40-700, 40-650, 40-600, 40-550, 40-500, 45-800, 45-750, 45-700, 45-650, 45-600, 45-550, or 45-500 nucleotides. Each possibility represents a separate embodiment of the invention. In some embodiments, the second strand comprises between 15-600 nucleotides. In some embodiments, the second strand comprises between 15-650 nucleotides. In some embodiments, the second strand comprises between 15-700 nucleotides.

In some embodiments, the support is a solid support. In some embodiments, the support is a surface. In some embodiments, the support is a bead. In some embodiments, the support is an artificial support. In some embodiments, the support is a man-made support. In some embodiments, the bead is a microbead. In some embodiments, the bead is a nanobead. In some embodiments, the support is a capture support. In some embodiments, the support is an amplification support. In some embodiments, the bead is a magnetic bead. In some embodiments, the bead is a paramagnetic bead. In some embodiments, the beads are capture beads.

The beads used herein may be of any convenient size and fabricated from any number of known materials. Example of such materials include: inorganics, natural polymers, and synthetic polymers. Specific examples of these materials include: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like, polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase supports known to those of skill in the art. In some embodiments, the bead is a Sepharose bead. In some embodiments, the beads are polystyrene beads. In some embodiments, the polystyrene is crosslinked polystyrene. In some embodiments, the polystyrene is a polystyrene core. In some embodiments, the beads are polyacrylamide beads. In some embodiments, the beads are polyacrylamide-coated beads. In some embodiments, the beads comprise a diameter of greater than about 0.1 μm, 0.2 μm, 0.3 μm, 0.4 μm, 0.5 μm, 0.6 μm, 0.7 μm, 0.8 μm, 0.9 μm, or 1 μm. Each possibility represents a separate embodiment of the invention. In some embodiments, the beads comprise a diameter of greater than about 0.2 μm. In some embodiments, the beads comprise a diameter of greater than about 0.5 μm. In some embodiments, the beads comprise a diameter of less than about 1, 1.5, 2, 2.5, 3, 3.5, 4, 4.5, 5, 5.5, 6, 6.5, 7, 7.5, 8, 8.5, 9, 9.5 or 10 μm. Each possibility represents a separate embodiment of the invention. In some embodiments, the beads comprise a diameter of less than about 2.5 μm. In some embodiments, the beads comprise a diameter of between about 0.1-5 μm, 0.1-4 μm, 0.1-3 μm, 0.1-2.5 μm, 0.1-2 μm, 0.1-1.5 μm, 0.1-1 μm, 0.2-5 μm, 0.2-4 μm, 0.2-3 μm, 0.2-2.5 μm, 0.2-2 μm, 0.2-1.5 μm, 0.2-1 μm, 0.3-5 μm, 0.3-4 μm, 0.3-3 μm, 0.3-2.5 μm, 0.3-2 μm, 0.3-1.5 μm, 0.3-1 μm, 0.4-5 μm, 0.4-4 μm, 0.4-3 μm, 0.4-2.5 μm, 0.4-2 μm, 0.4-1.5 μm, 0.4-1 μm, 0.5-5 μm, 0.5-4 μm, 0.5-3 μm, 0.5-2.5 μm, 0.5-2 μm, 0.5-1.5 μm, or 0.5-1 μm. Each possibility represents a separate embodiment of the invention. In some embodiments, the beads comprise a diameter of between about 0.2-2.5 μm. In some embodiments, the beads comprise a diameter of between about 0.5-2.5 μm. In some embodiments, the beads are microbeads. In some embodiments, the beads are nanobeads.

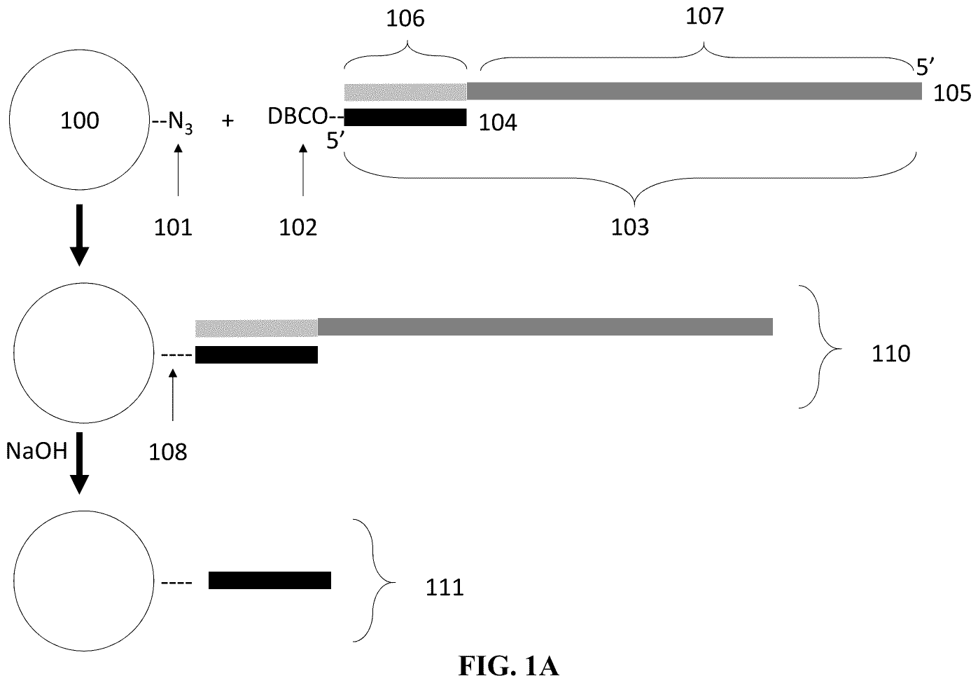

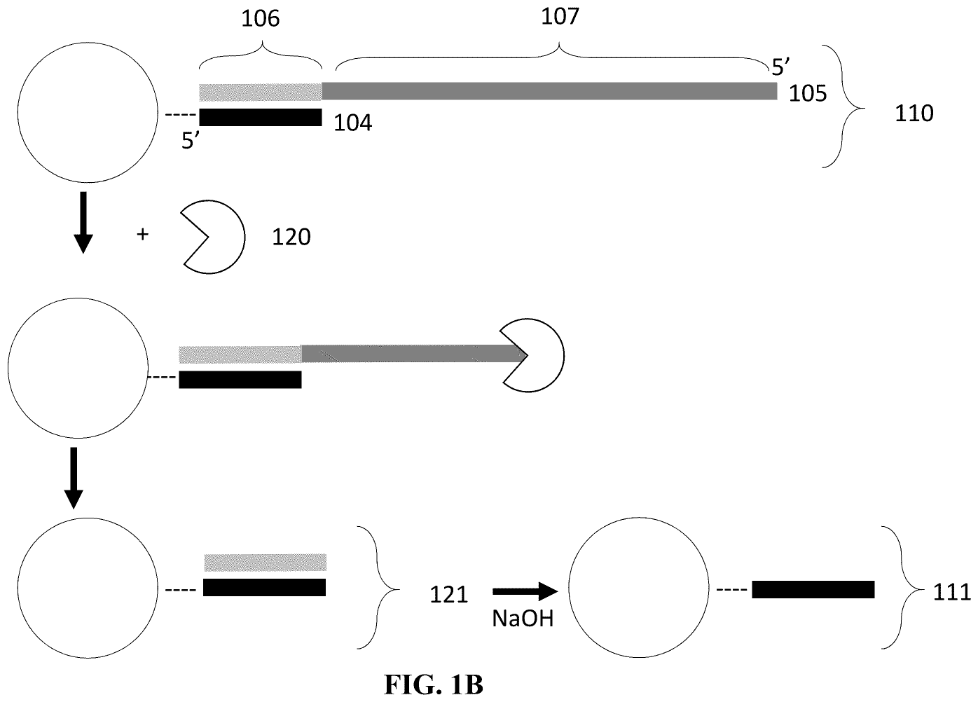

Reference is now made to FIG. 1A. Support 100 is shown here as a bead, though it will be understood that it can be any support known in the art, e.g., a solid support. A single support is shown for simplicity, but it will be understood that a plurality of supports can be used. In some embodiments, a support is a plurality of supports. First coupling moiety 101 is located on the surface of support 100. In some embodiments, the support comprises a plurality of first coupling moieties. In some embodiments, the first coupling moiety is a functional group. The support may be functionalized, or an already functionalized support may be provided. In some embodiments, the functional group is an azide. In some embodiments, the first coupling moiety is an azide. In some embodiments, the first coupling moiety is a moiety capable of coupling to a second coupling moiety. In some embodiments, the first coupling moiety is a moiety capable of or configured for forming a covalent bond with a second coupling moiety. In some embodiments, the first coupling moiety is capable of coupling (e.g., coupling to a second coupling moiety) by click chemistry or by a click reaction. In some embodiments, the first coupling moiety is not a nucleic acid molecule. In some embodiments, the first coupling moiety is not a nucleotide. In some embodiments, the first coupling moiety is a part of the support. In some embodiments, the first coupling moiety is directly linked to the solid support. In some embodiments, solid support is functionalized with the first coupling moiety.

Coupling pairs that may make up the first and second coupling moieties are well known in the art. In some embodiments, the coupling between the first and second coupling moieties produces a covalent bond. Examples of coupling pairs include, but are not limited to, biotin-avidin, carboxylic acid-amino group, NHS ester-amino group, maleimide-thiol, and Azide-DBCO. Azide to DBCO coupling is exemplified herein, but is only one capture option, and any such capture system known in the art may be employed.

In this embodiment, first coupling moiety 101 is shown as an azide (N₃) moiety. Azide functionalization of beads is well known in the art, and it will be understood that any method of functionalizing or, alternatively, any pre-functionalized support may be used. The actual linkage made to link the nucleic acid to the support is secondary to the makeup of the nucleic acid molecule which controls the density/number of molecules that can be conjugated to a given support. The first coupling moiety 101 reacts with the second coupling moiety 102. In this embodiment, the second coupling moiety is shown as a dibenzocyclooctyne group (DBCO). Azide moieties react with diarylcyclooctyne moieties in a copper-free click reaction to form a covalent bond. In some embodiments, the second coupling moiety is a diarylcyclooctyne moiety. In some embodiments, the diarylcyclooctyne is DBCO. It will be understood that the first and second coupling moieties can be reversed and the DBCO could be on the support while the azide could be located on the nucleic acid molecule.

In some embodiments, the support, e.g., the solid support, comprises at least about 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000 or 300,000 first coupling moieties. Each possibility represents a separate embodiment of the invention. In some embodiments, the support comprises at least 150,000 first coupling moieties. In some embodiments, the support comprises a density of at least about 10,000, 20,000, 30,000, 40,000, 50,000, 60,000, 70,000, 80,000, 90,000, or 100,000 first coupling moieties per cubic micron. Each possibility represents a separate embodiment of the invention. In some embodiments, the support comprises a density of at least about 50,000 first coupling moieties per cubic micron. In some embodiments, the support comprises a density of at least about 10,000, 20,000, 30,000, 40,000, 50,000, 60,000, 70,000, 80,000, 90,000, or 100,000 first coupling moieties per square micron of support surface. Each possibility represents a separate embodiment of the invention. In some embodiments, the support comprises a density of at least about 50,000 first coupling moieties per square micron of support surface.

As shown in FIG. 1A, support 100 is contacted with nucleic acid molecule 103. It will be understood that in fact a plurality of nucleic acid molecules is provided, but for simplicity only a single one is shown. In some embodiments, the support and nucleic acid molecule are contacted under conditions sufficient to couple the first and second coupling moieties. In some embodiments, the conditions are conditions sufficient to couple the first coupling moiety to the second coupling moiety. Coupling reactions are well known in the art, as are conditions sufficient and/or required for that coupling. For example, a copper click reaction would need to be carried out in conditions that comprise copper. Similarly, the solutions selected, ionic content, temperature, acidity, etc. may all be modified to facilitate coupling of particular first and second coupling moieties.

Nucleic acid molecule 103 comprises a first strand 104 and a second strand 105. In some embodiments, each nucleic acid molecule comprises a double-stranded region. In some embodiments, the double-stranded region comprises a region of the first and a region of the second strands. In some embodiments, the region of the first strand is the entire first strand. Nucleic acid molecule 103 comprises a second coupling moiety 102. Second coupling moiety 102 is configured to couple to first coupling moiety 101. Second coupling moiety 102 is located on first strand 104, at the 5′ end of first strand 104. It will be understood that the second coupling moiety could also be located at the 3′ end of the first strand or indeed within the middle of the strand. For simplicity, all figures show a 5′ linked second coupling moiety. Functionalization of nucleic acid molecules with reactive coupling groups is well known in the art. Internal nucleotides can also be used for coupling. In some embodiments, the second coupling moiety is at a 5′ end of the first strand. In some embodiments, the second coupling moiety is at a 3′ end of the first strand. In some embodiments, an end is a terminal nucleotide. In some embodiments, the second coupling moiety is linked to an internal nucleotide of the first strand.

In some embodiments, the first strand is a primer. In some embodiments, the first strand is a capture oligonucleotide. It will be understood by a skilled artisan that the first strand is intended to hybridize to a target sequence on a nucleic acid molecule of interest. In some embodiments, the first strand is complementary to a sequence on a nucleic acid molecule of interest. In some embodiments, the sequence is a target sequence. In some embodiments, the nucleic acid molecule of interest is a target molecule. In some embodiments, the sequence is a universal sequence. In some embodiments, the universal sequence is a sequence universal to a library (e.g., a library of nucleic acid molecules). In some embodiments, the library is a library of target molecules. In some embodiments, the target molecule is single-stranded. In some embodiments, the target molecule is a molecule to be amplified.

Binding of single-stranded primers to supports to generate a capture substrate is well known in the art. However, in the present disclosure a bulky group molecule is present in addition to the primer (e.g., bound or hybridized to the primer). This steric bulk imposes physical constraints on the distance between primers and effectively forces the primers to be more spaced apart on the support. This spacing leads to an overall reduction in the density of primers on the support, but does not impair subsequent enzymatic reactions (e.g., amplification) using the primers.

In FIG. 1A the bulky group is second strand 105 which is hybridized to first strand (e.g., primer) 104, resulting in nucleic acid molecule 103. Second strand 105 comprises region 106 that has reverse complementarity to first strand 104. Region 106 is hybridized to first strand 104. This hybridization and complementarity can be perfect complementarity or there can be one or more mismatches, so long as the two strands remain hybridized during coupling of nucleic acid molecule 103 to support 100. Region 106 could even be shorter than first strand 104 with some nucleotides absent so long as hybridization remains during coupling of nucleic acid molecule 103 to support 100.

Second strand 105 may also comprise region 107, which is not complementary or hybridized to first strand 104. In this embodiment, region 106 is 3′ to region 107; however, the reverse is also envisioned. It is the physical bulk of second strand 105 and, in particular, the bulk of region 107 that forces distance between conjugating molecules. As such the effect is not sequence specific. In some embodiments, the region that is not hybridized comprises a random sequence. In some embodiments, the region that is not hybridized comprises a naturally occurring sequence that is not of interest. Sequences may be selected that do not comprise large amounts of secondary structure (e.g., sequencing lacking substantial secondary structure) or that lack hairpin regions, although this is not essential. Similarly, the exact position of this bulk within the second strand is not essential, and the unhybridized region 107 may be 5′ or 3′ or both 5′ and 3′ (e.g., a noncontiguous unhybridized region) to region 106. However, in preferred implementations, there is a contiguous unhybridized region disposed 5′ to region 106.

If the unhybridized region is both 5′ and 3′ primer to region 106, then there would in effect be two regions 107.

In some embodiments, the region of the second strand not hybridized to the first strand comprises at least about 0, 5, 10, 15, 20, 30, 40, 50, 60, 70, 80, 90, 100, 120, 130, 140, 150, 160, 170, 180, 190, 200, 250, 300, 350, 400, 450, or 500 nucleotides. In some instances, the region of the second strand not hybridized to the first strand comprises at least 100 nucleotides. In some embodiments, the region of the second strand not hybridized to the first strand comprises at least 500 nucleotides. In some instances, the region of the second strand not hybridized to the first strand comprises at most about 400, 450, 500, 550, 600, 650, 700, 750, 800, 850, 900, 950, or 1000 nucleotides. In some instances, the region of the second strand not hybridized to the first strand comprises at most 650 nucleotides. In some embodiments, the region of the second strand not hybridized to the first strand comprises at most 700 nucleotides. In some embodiments, the region of the second strand not hybridized to the first strand comprises between 20-800, 20-750, 20-700, 20-650, 20-600, 20-550, 20-500, 50-800, 50-750, 50-700, 50-650, 50-600, 50-550, 50-500, 100-800, 100-750, 100-700, 100-650, 100-600, 100-550, 100-500, 200-800, 200-750, 200-700, 200-650, 200-600, 200-550, 200-500, 300-800, 300-750, 300-700, 300-650, 300-600, 300-550, 300-500, 400-800, 400-750, 400-700, 400-650, 400-600, 400-550, 400-500, 500-800, 500-750, 500-700, 500-650, 500-600 or 500-550 nucleotides. Each possibility represents a separate embodiment of the invention. In some embodiments, the region of the second strand not hybridized to the first strand comprises between 100-600 nucleotides. In some embodiments, the region of the second strand not hybridized to the first strand comprises between 100-650 nucleotides. In some embodiments, the region of the second strand not hybridized to the first strand comprises between 100-700 nucleotides. In some embodiments, the region of the second strand not hybridized to the first strand comprises between 500-600 nucleotides. In some embodiments, the region of the second strand not hybridized to the first strand comprises between 500-650 nucleotides. In some embodiments, the region of the second strand not hybridized to the first strand comprises between 500-700 nucleotides.

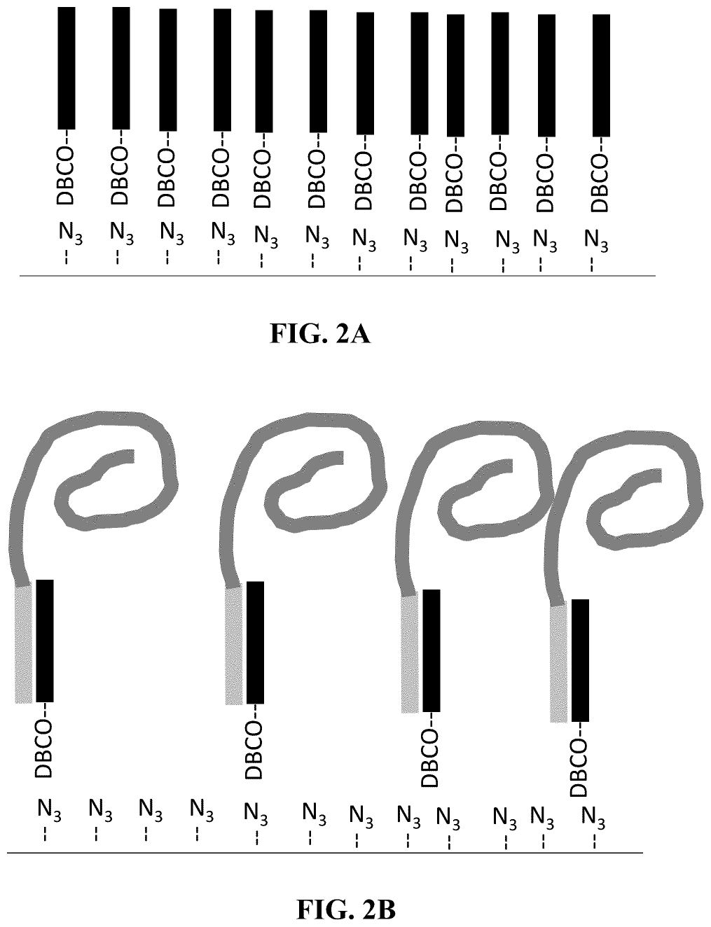

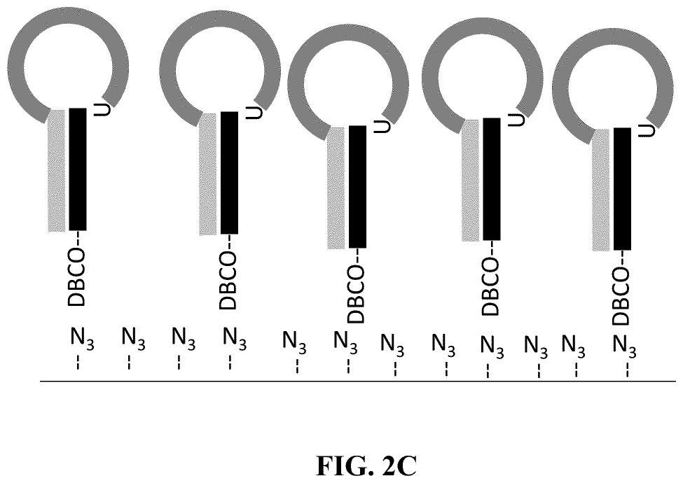

The effect of this configuration of the nucleic acid molecule can be seen in FIGS. 2A-2B. The standard method of generating a capture bead is shown in FIG. 2A. Azide molecules are distributed essentially evenly over the surface of the support and DBCO conjugated short single-stranded primers are able to reach and bind to essentially every azide. In FIG. 2B, the same support with evenly distributed azides is present; however, the bulk of the second strand unhybridized region (or the polymer, see hereinbelow) blocks access to many of the azides. The result is that the density of nucleic acid molecules bound to the support is greatly reduced. It will be apparent that the size of the bulky molecule will be inversely proportionate to the density of the nucleic acid molecules on the support. Thus, by altering the size of the bulky molecule (increasing/decreasing the length of the unhybridized region or polymer) one can control the density on the support.

Bringing nucleic acid molecule 103 into contact with support 100, under conditions sufficient for coupling of first coupling moiety 101 to second coupling moiety 102, results in the production of coupled support 110. Within coupled support 110, the first and second coupling moieties have reacted to produce linkage 108. In this embodiment, linkage 108 is a covalent bond. It will be understood that the type of linkage formed is dependent on the pair of coupling moieties used, and that any such possible linkage is envisioned as part of the invention.

Following the conjugation, any non-reacted nucleic acid molecules 103 can be removed from the reaction and potentially recycled in a future coupling. This can be facilitated by isolating coupled support 110, e.g., by applying a magnetic field to a paramagnetic bead. This leaves a population of solid supports coupled to nucleic acid molecules. Each solid support of the population is coupled to nucleic acid molecules at less than a maximal density. The maximal density being the density achieved when only the first strand alone is coupled to the support. In some embodiments, the maximal density comprises all first coupling moieties coupled to a nucleic acid molecule. In some embodiments, the density is the percent occupancy of the first coupling moieties by nucleic acid molecules. In some embodiments, the maximal density comprises the maximum number of first coupling moieties on a given support that can couple to any nucleic acid molecule being coupled. In some embodiments, the maximal density comprises the maximum number of first coupling moieties on a given support that can couple to any nucleic acid molecule of the same length as the first strand being coupled. It will be understood that the maximum number of bound molecules will depend on the molecule being bound. For short single-stranded molecules, the maximum number that can be bound is essentially equal to the number of first coupling moieties. In some embodiments, short is equal to or less than 15, 20, 25, 30, 35, 40, or 45 nucleotides in length. Each possibility represents a separate embodiment of the invention. In some embodiments, short is equal to or less than 15 nucleotides in length. In some embodiments, short is equal to or less than 45 nucleotides in length.

In some embodiments, less than maximal density is a predetermined percentage of maximal density. In some embodiments, less than maximal density is less than a predetermined percentage of maximal density. In some embodiments, the predetermined percentage is about 20%, about 25%, about 30%, about 40%, about 50%, about 60%, about 70%, about 75%, about 80%, or about 90%. Each possibility represents a separate embodiment of the invention. In some embodiments, the predetermined percentage is about 50%. In some embodiments, less than 50% of maximal density comprises at least half of said first coupling moieties not coupled to a nucleic acid molecule.

In some embodiments, the coupled solid support comprises first coupling moieties coupled to the second coupling moiety and first coupling moieties not coupled to the second coupling moiety. In some embodiments, the distance between adjacent first coupling moieties coupled to the second coupling moiety is at least about 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, or 20 nm. Each possibility represents a separate embodiment of the invention. In some embodiments, the distance is the average distance. In some embodiments, average distance is average distance on the support (e.g., on the surface of the support or within the volume of the support). In some embodiments, the distance between adjacent first coupling moieties coupled to the second coupling moiety is at least about 2 nm. In some embodiments, the distance between adjacent first coupling moieties coupled to the second coupling moiety is at least about 10 nm. In some embodiments, the coupled solid support comprises at least one first coupling moiety coupled to a second coupling moiety that is at least about 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, or 20 nm distant from another first coupling moiety coupled to a second coupling moiety. Each possibility represents a separate embodiment of the invention. In some embodiments, the coupled solid support comprises at least one first coupling moiety coupled to a second coupling moiety that is at least about 2 nm distant from any other first coupling moieties coupled to a second coupling moiety. In some embodiments, the coupled solid support comprises at least one first coupling moiety coupled to a second coupling moiety that is at least about 10 nm distant from any other first coupling moieties coupled to a second coupling moiety.

In some embodiments, the average distance between a first first coupling moiety and second first coupling moiety is less than about 0.1 nm, 0.2 nm, 0.3 nm, 0.4 nm, 0.5 nm, 0.6 nm, 0.7 nm, 0.8 nm, 0.9 nm, 1 nm, 1.1 nm, 1.2 nm, 1.3 nm, 1.4 nm, 1.5 nm, 1.6 nm, 1.7 nm, 1.8 nm, 1.9 nm, 2 nm, 2.1 nm, 2.2 nm, 2.3 nm, 2.4 nm, 2.5 nm, 2.6 nm, 2.7 nm, 2.8 nm, 2.9 nm, or 3 nm. Each possibility represents a separate embodiment of the invention. In some embodiments, the average distance between a first first coupling moiety and second first coupling moiety is less than about 2 nm. In some embodiments, the distance between an average first first coupling moiety and second first coupling moiety is less than about 1 nm.

In some embodiments, the solid support comprises at least about 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000 or 300,000 first coupling moieties. Each possibility represents a separate embodiment of the invention. In some embodiments, the solid support comprises at least about 150,000 first coupling moieties. In some embodiments, the solid support comprises at least about 200,000 first coupling moieties.

In some embodiments, each solid support is coupled to not more than about 80,000, 75,000, 70,000, 65,000, 60,000, 55,000, 50,000, 45,000, 40,000, 35,000, 30,000, 25,000, 20,000, 15,000, or 10,000 nucleic acid molecules. Each possibility represents a separate embodiment of the invention. In some embodiments, each solid support is coupled to not more than about 75,000 nucleic acid molecules. In some embodiments, each solid support is coupled to not more than about 50,000 nucleic acid molecules. In some embodiments, each solid support is coupled to not more than about 10,000 nucleic acid molecules. In some embodiments, not more than is less than. In some embodiments, the threshold number is about 80,000, 75,000, 70,000, 65,000, 60,000, 55,000, 50,000, 45,000, 40,000, 35,000, 30,000, 25,000, 20,000, 15,000, or 10,000 nucleic acid molecules. Each possibility represents a separate embodiment of the invention. In some embodiments, the threshold number is about 75,000. In some embodiments, the threshold number is about 50,000. In some embodiments, the threshold number is about 10,000. In some embodiments, the threshold number is less than the maximum. In some embodiments, the maximum is the maximum number of nucleic acid molecules that can be coupled to the solid support. In some embodiments, the maximum is the maximum number of nucleic acid molecules of the same length as the first strand that can be coupled to the solid support.

In some embodiments, each solid support is coupled to nucleic acid molecules at a density of not more than about 175,000, 170,000, 165,000, 160,000, 155,000, 150,000, 145,000, 140,000, 135,000, 130,000, 125,000, 120,000, 115,000, 110,000, 105,000, 100,000, 95,000, 90,000, 85,000, 80,000, 75,000, 70,000, 65,000, 60,000, 55,000, 50,000, 45,000, 40,000, 35,000, 30,000, 25,000, 20,000, 15,000, 10,000, 7,500, 5,000 or 2,500 nucleic acid molecules per cubic micron. Each possibility represents a separate embodiment of the invention. In some embodiments, the density that each coupled solid support is coupled to nucleic acid molecules is less than about 75,000 nucleic acid molecules per cubic micron. In some embodiments, the density that each coupled solid support is coupled to nucleic acid molecules is less than about 20,000 nucleic acid molecules per square micron.

In some embodiments, each solid support is coupled to not more than about 160,000 nucleic acid molecules, not more than about 125,000 nucleic acid molecules, not more than about 75,000 nucleic, not more than about 50,000 nucleic acid molecules, or not more than about 10,000 nucleic acid molecules. In some embodiments, not more than is less than.

Once free nucleic acid molecules are removed, second strand 105 can be denatured from first strand 104. Denaturing of complementary strands can be performed by any method known in the art, including heating or sodium hydroxide treatment for example. In some embodiments, the denaturation is chemical denaturation. In some embodiments, the denaturation is thermal denaturation. In some embodiments, the denaturation is with sodium hydroxide. The denaturing results in a support coupled to a single-stranded primer 111. In some embodiments, the denaturing produces a population of solid supports coupled to a plurality of single-stranded primers. In some embodiments, the method further comprises denaturing the second strand from the first strand. In some embodiments, denaturing comprises subjecting the double-stranded region to conditions sufficient to denature the second strand from the first strand. In some embodiments, denaturing comprises subjecting the coupled support to conditions sufficient to denature the second strand from the first strand. In some embodiments, the denaturing results in a solid support coupled to a plurality of single-stranded primers. In some embodiments, the denaturing is complete denaturing. In some embodiments, the denaturing removes the second strand from the coupled support. The supports coupled to single-stranded primers are now ready for downstream reactions, such as capturing a template molecule and clonal amplification of that captured molecule. The step of denaturation can be performed just before the capture is to be performed. That is, the capture substrates can be stored for a period of time between its generation and its eventual use (e.g., during transport from a manufacturing site to an operating site, sample processing and preparation, etc.) with a second strand still hybridized. In some embodiments, a capture substrate hybridized to one or more second strands may be stored for at least 1 hour, and in some cases may be stored for months or even years.

Unhybridized region 107 may be removed before capture of target (e.g., template) molecules and subsequent enzymatic procedures. This may be important because unhybridized region 107 is essentially a mock-template molecule. Second strand 105 contains the target sequence that hybridizes to the single-stranded primer (the sequence or region 106) and contains a sequence that could potentially be amplified (the sequence of region 107). It is region 107 that is of particular concern; if only region 106 were present, while it would block binding of target molecules, it could not lead to erroneous amplification. Denaturation of second strand 105 may solve this problem but runs the risk that some strands may not fully denature away or may rehybridize after denaturation. Reference is now made to FIG. 1B which provides a solution to this problem. FIG. 1B shows a variation of the method presented in FIG. 1A but that ensures removal of region 107. Coupled support 110 is produced as described hereinabove and is treated with a 5′ to 3′ single-stranded nuclease 120, such as Mung Bean Nuclease. In some embodiments, another single-stranded (e.g., ssDNA or RNA) specific endonuclease can be used instead. Nuclease 120 digests region 107 but cannot digest region 106 and cannot digest first strand 104. This essentially blunts second strand 105 and leaves only first strand 104 hybridized to region 106. It will be understood by a skilled artisan that if region 107 were a 3′ overhang than a 3′ to 5′ single-stranded exonuclease would be employed. The result of this digestion is a coupled support 121 which comprises support 100 coupled to first strand 104 and only region 106 of second strand 105. Denaturation of coupled support 121 results in a support coupled to a single-stranded primer 111, which is the same end product as the method provided in FIG. 1A.

As before, the denaturation step can be performed immediately before binding of target molecules. Coupled support 121 can therefore be stored as is with double-stranded molecules on the surface of the supports. This method of storage is advantageous as it reduces bead clumping and undesired interaction between individual beads. The single-stranded molecules on the supports are generally not homologous to each other and should not hybridize, nevertheless some interaction between single-stranded molecules does occur. This can lead to clumping during storage. Supports comprising only double-stranded molecules are less likely to clump. With no free single strands (e.g., no unhybridized primers), no base-pairing is possible between the beads. Thus, this method provides the added benefit of a superior storage solution and no risk of leftover mock-template that could interfere with later enzymatic steps.

Nucleases are well known in the art and are commercially available. Further, several known nucleases comprise 5′ to 3′ and/or 3′ to 5′ activity. In some embodiments, the nuclease is an exonuclease. In some embodiments, the nuclease is an endonuclease. In some embodiments, the nuclease is a single-stranded nuclease. In some embodiments, the nuclease does not digest double-stranded nucleic acids. In some embodiments, the nuclease is a DNA nuclease. In some embodiments, the nuclease is an RNA nuclease. In some embodiments, the nuclease is a polymerase. In some embodiments, the nuclease is not a polymerase. In some embodiments, the exonuclease is a class I exonuclease and comprises 3′ to 5′ nuclease activity. In some embodiments, the exonuclease is a class II exonuclease and comprises 5′ to 3′ nuclease activity. In some embodiments, the exonuclease is a class IV exonuclease and comprises 5′ to 3′ nuclease activity. In some embodiments, the exonuclease is a class V exonuclease and comprises 3′ to 5′ nuclease activity. In some embodiments, the exonuclease is a class VII exonuclease and comprises 5′ to 3′ and 3′ to 5′ nuclease activity. Examples of 5′ to 3′ nucleases that cleave only single-stranded nucleic acids include, but are not limited to, RecJf, Mung Bean nuclease, Exonuclease VII, and nuclease P1. In some embodiments, the 5′ to 3′ nuclease is Mung Bean nuclease. Examples of 3′ to 5′ nucleases that cleave only single-stranded nucleic acids include, but are not limited to, Exonuclease I, Exonuclease T and Exonuclease VII.

In some embodiments, the method comprises contacting the nucleic acid molecule with a nuclease. In some embodiments, the nuclease is capable of degrading a 5′ unhybridized region of the second strand. In some embodiments, the nuclease is capable of degrading a 3′ unhybridized region of the second strand. In some embodiments, the contacting of the nuclease is under conditions suitable for nuclease activity. In some embodiments, the conditions are suitable for 5′ to 3′ nuclease activity. In some embodiments, the conditions are suitable for 3′ to 5′ nuclease activity. In some embodiments, the conditions are suitable for nuclease activity against single-stranded nucleic acids. In some embodiments, the conditions are not suitable for nuclease activity against double-stranded nucleic acids. In some embodiments, the degrading leaves a hybridized region of the second strand still hybridized to the first strand. In some embodiments, nuclease is not capable of degrading the hybridized region of the first strand and second strand. In some embodiments, the nuclease is not capable of degraded double-stranded nucleic acids.

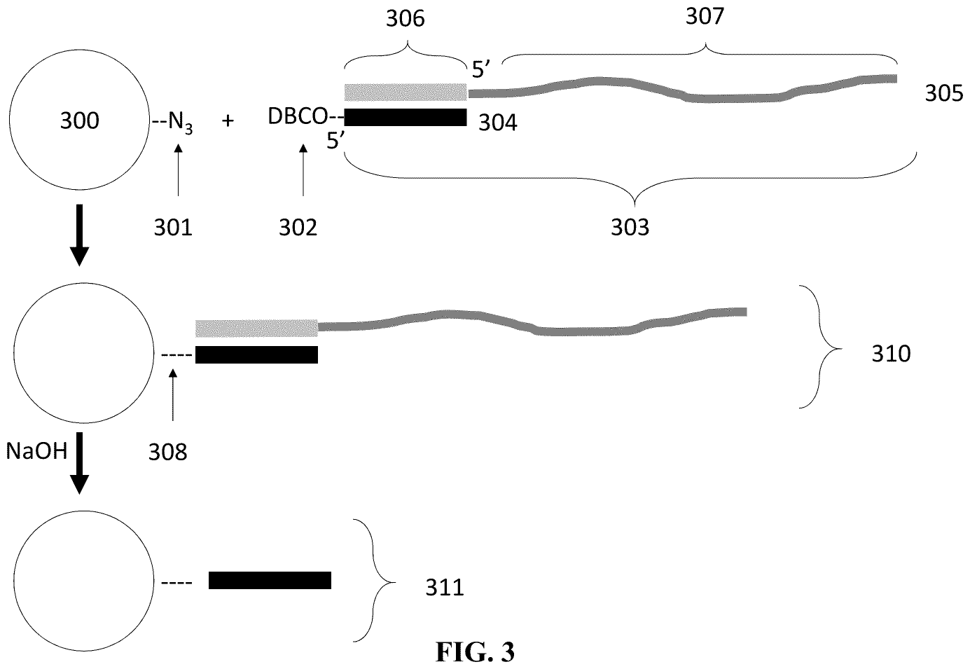

Reference is now made to FIG. 3. FIG. 3 shows an alternative embodiment in which the bulky group of the second strand is a non-nucleotide polymer. Support 300 comprises first coupling moiety 301 which is equivalent to support 100 and first coupling moiety 101 of FIG. 1A. Nucleic acid molecule 303 comprises first strand 304 and second strand 305, where first strand 304 comprises a 5′ second coupling moiety 302. Second strand 305 comprises region 306, which is complementary and hybridized to first strand 304. Second strand 305 further comprises a 5′ linked polymer 307. This polymer acts much the same way as the 5′ overhang (region 107) of FIG. 1A does. It forms a random coil in solution that may occlude available azides on the support, thus forcing the next nucleic acid molecule to bind farther away and overall reducing the density of nucleic acid molecules bound to the support (see, for example, FIG. 2B).

Coupling of first coupling moiety 301 to second coupling moiety 302 links nucleic acid molecule 303 to support 300 via bond 308. The result is coupled support 310. Second strand 305 can be denatured from first strand 304 to produce a support coupled to a single-stranded primer 311 which is the same as support coupled to a single-stranded primer 111.

In some embodiments, the polymer comprises a regularly repeating unit. In some embodiments, the polymer comprises a copolymer. In some embodiments, the copolymer comprises two or more repeating units, wherein at least one of the two or more repeating units is an amino acid. In some embodiments, the amino acid is a non-proteinogenic amino acid. In some embodiments, the copolymer does not comprise a regularly repeating unit. In some embodiments, the copolymer is a block copolymer.

In some embodiments, the polymer is a homopolymer. In some embodiments, the polymer is a linear polymer. In some embodiments, the polymer is a branched polymer. In some embodiments, the polymer is a block polymer. In some embodiments, the polymer is a synthetic polymer. In some embodiments, the polymer is an organic polymer. In some embodiments, the polymer is an amphiphilic polymer. In some embodiments, the second strand is a DNA block copolymer (DBC).

Polymers and copolymers are well known in the art, and include, for example, polyethylene glycol (PEG), polyethyleneimine (PEI), poly-L-glutamate (poly(L-glu)), poly-proline, poly(acrylamide), poly(dimethylacrylamide), poly(acrylic acid), poly(methacrylic acid), poly(2-hydroxyethylacrylate), poly(2-hydroxyethyl acrylamide), dextran, chitosan, and poly(hyaluronic acid). In some embodiments, the polymer is PEG polymer. In some embodiments, the polymer is a poly-(L-glu) polymer. In some embodiments, the polymer does not comprise a strong positive charge. In some embodiments, the polymer does not comprise a stretch of positive charges. It will be understood by a skilled artisan that a strongly positive polymer will bind to negatively charged nucleic acids, this may result in clumping of supports and should be avoided. In some embodiments, the polymer is not PEI. In some embodiments, the polymer is a neutral polymer. In some embodiments, the polymer is an inert polymer.

Methods of linking nucleic acids to polymers are well known in the art and any such linkage may be employed. Examples of such methods can be found for example in Zhang et al., 2015, “Biodegradable DNA-Brush Block Copolymer Spherical Nucleic Acids Enable Transfection Agent-Free Intracellular Gene Regulation”, Small, 11:5360-5368; Bilgic and Klok, 2015, “Oligonucleotide Immobilization and Hybridization on Aldehyde-Functionalized Poly(2-hydroxyethyl methacrylate) Brushes”, Biomacromolecules, 16, 11, 3657-3665; and Amblard et al., 2009, “Cu(I)-Catalyzed Huisgen Azide-Alkyne 1,3-Dipolar Cycloaddition Reaction in Nucleoside, Nucleotide, and Oligonucleotide Chemistry”, Chem Rev., 109(9): 4207-4220 the contents of which are herein incorporated by reference in their entirety. In some embodiments, the linkage is a covalent bond. In some embodiments, the linkage is formed by a click reaction. For example, the nucleic acid molecule that is the second strand may be generated with a DBCO moiety at the 5′ end and the polymer may have an azide group at one terminus. The click reaction will result in a copolymer of DNA and the polymer such as may be used in a method of the invention.

In some embodiments, the polymer may have a size of at least about 1,000, 2,000, 3,000, 4,000, 5000, 6,000, 7,000, 8,000, 9,000, 10,000, 15,000, 20,000, 25,0000, 30,000, 35,000, 40,000, 45,000, 50,000, 60,000, 70,000, 80,000, 90,000, or 100,000 g/mol. Each possibility represents a separate embodiment of the invention. In some embodiments, the polymer may have a size of at least about 10,000 g/mol. In some embodiments, the polymer may have a size of at least about 100,000 g/mol. In some embodiments, the polymer may have a size of at most about 100,000, 150,000, 200,000, 250,0000, 300,000, 350,000, 400,000, 450,000, 500,000, 550,000, 600,000, 650,000, 700,000, 750,000, 800,000, 850,000, 900,000, 950,000, or 1,000,000 g/mol. Each possibility represents a separate embodiment of the invention. In some embodiments, the polymer may have a size of at most 700,000 g/mol. In some embodiments, the polymer may have a size of between about 5,000-500,000, 5,000-550,000, 5,000-600,000, 5,000-650,000, 5,000-700,000, 5,000-750,000, 5,000-800,000, 5,000-850,000, 5,000-900,000, 10,000-650,000, 10,000-700,000, 10,000-750,000, 10,000-800,000, 10,000-850,000, 10,000-900,000, 15,000-650,000, 15,000-700,000, 15,000-750,000, 15,000-800,000, 15,000-850,000, 15,000-900,000, 25,000-650,000, 25,000-700,000, 25,000-750,000, 25,000-800,000, 25,000-850,000, 25,000-900,000, 50,000-650,000, 50,000-700,000, 50,000-750,000, 50,000-800,000, 50,000-850,000, 50,000-900,000, 75,000-650,000, 75,000-700,000, 75,000-750,000, 75,000-800,000, 75,000-850,000, 75,000-900,000, 100,000-650,000, 100,000-700,000, 100,000-750,000, 100,000-800,000, 100,000-850,000, or 100,000-900,000 g/mol. Each possibility represents a separate embodiment of the invention. In some embodiments, the polymer may have a size of between about 10,000-700,000 g/mol. In some embodiments, the polymer is PEG20k.

In some embodiments, the polymer may have a radius of about 1-100 nm, 1-75 nm, 1-50 nm, 1-45 nm, 1-40 nm, 1-35 nm, 1-30 nm, 1-25 nm, 1-20 nm, 1-15 nm, 1-10 nm, 1-9 nm, 1-8 nm, 1-7 nm, 1-6 nm, 1-5 nm, 1-4 nm, 1-3 nm, 1-2 nm, 2-100 nm, 2-75 nm, 2-50 nm, 2-45 nm, 2-40 nm, 2-35 nm, 2-30 nm, 2-25 nm, 2-20 nm, 2-15 nm, 2-10 nm, 2-9 nm, 2-8 nm, 2-7 nm, 2-6 nm, 2-5 nm, 2-4 nm, 2-3 nm, 3-100 nm, 3-75 nm, 3-50 nm, 3-45 nm, 3-40 nm, 3-35 nm, 3-30 nm, 3-25 nm, 3-20 nm, 3-15 nm, 3-10 nm, 3-9 nm, 3-8 nm, 3-7 nm, 3-6 nm, 3-5 nm, 3-4 nm, 4-100 nm, 4-75 nm, 4-50 nm, 4-45 nm, 4-40 nm, 4-35 nm, 4-30 nm, 4-25 nm, 4-20 nm, 4-15 nm, 4-10 nm, 4-9 nm, 4-8 nm, 4-7 nm, 4-6 nm, 4-5 nm, 5-100 nm, 5-75 nm, 5-50 nm, 5-45 nm, 5-40 nm, 5-35 nm, 5-30 nm, 5-25 nm, 5-20 nm, 5-15 nm, 5-10 nm, 5-9 nm, 5-8 nm, 5-7 nm, or 5-6 nm. Each possibility represents a separate embodiment of the invention. In some embodiments, the polymer may have a radius of about 1-3 nm. In some embodiments, the polymer may have a radius of about 5-100 nm. In some embodiments, the radius is a radius of gyration. In some embodiments, the radius is a hydrodynamic radius.

The use of a polymer as a bulky group also solves the problem of the risk of amplification from a mock-template molecule used as the bulky group Amplification may not proceed from a non-nucleotide polymer. Further, if the polymer is retained during storage, it may also inhibit clumping between beads. Lastly, the second strand with a polymer offers the added benefit of being recyclable. After denaturation of the second strand from the first strand the second strand can be collected from solution by isolation of the polymer. The second strand can then be rehybridized to new first strand and the resultant double-stranded molecule with a polymer can be reused in a method of the invention. In some embodiments, the method further comprises after denaturing the second strand from the first strand collecting the second strands and reusing them in a method of the invention. In some embodiments, the collecting is by collecting the polymer. In some embodiments, the collecting is by isolating the polymer.

By another aspect, there is provided a method of controlling density of nucleic acid molecules coupled to supports, the method comprising: (a) providing a plurality of supports, wherein each support comprises a plurality of first coupling moieties; and (b) contacting the plurality of supports with a plurality of nucleic acid molecules under conditions sufficient to couple the first coupling moiety to a second coupling moiety of the plurality of nucleic acid molecules to produce a coupled solid support, wherein each nucleic acid molecule comprises a single strand comprising a double-stranded region in which a first region of the single strand is hybridized to a second region of the single strand and wherein the single strand comprises a second coupling moiety configured to couple to the first coupling moiety, thereby producing a population of coupled solid supports that is coupled to nucleic acid molecules, wherein each coupled solid support is coupled to the nucleic acid molecules at a density that is less than a maximal density.

By another aspect, there is provided a method of controlling an amount of nucleic acid molecules coupled to supports, the method comprising: (a) providing a plurality of supports, wherein each support comprises a plurality of first coupling moieties; and (b) contacting the plurality of supports with a plurality of nucleic acid molecules under conditions sufficient to couple the first coupling moiety of said plurality of first coupling moieties to a second coupling moiety of the plurality of nucleic acid molecules to produce a coupled solid support, wherein each nucleic acid molecule comprises a strand comprising a double-stranded region in which a first region of the strand is hybridized to a second region of the strand and wherein the strand comprises the second coupling moiety configured to couple to the first coupling moiety, thereby producing a population of coupled solid support that is coupled to nucleic acid molecules, wherein each coupled solid support is coupled to fewer than a threshold number of nucleic acid molecules.

In some embodiments, the strand is a single strand. In some embodiments, the strand is a third strand. In some embodiments, the nucleic acid molecule is a single-stranded molecule. In some embodiments, the single-stranded molecule comprises a double-stranded region. In some embodiments, the first region of the third strand is a 5′ region of the third strand and the second region of the third strand is a 3′ region of the third strand. In some embodiments, the nucleic acid molecule comprises a single-stranded region between the first region and the second region. In some embodiments, the nucleic acid molecule is a hairpin. In some embodiments, the nucleic acid molecule comprises a third region between the first region and the second region. In some embodiments, the third region is an unhybridized region. In some embodiments, the unhybridized region comprises at least one cleavable or excisable base.

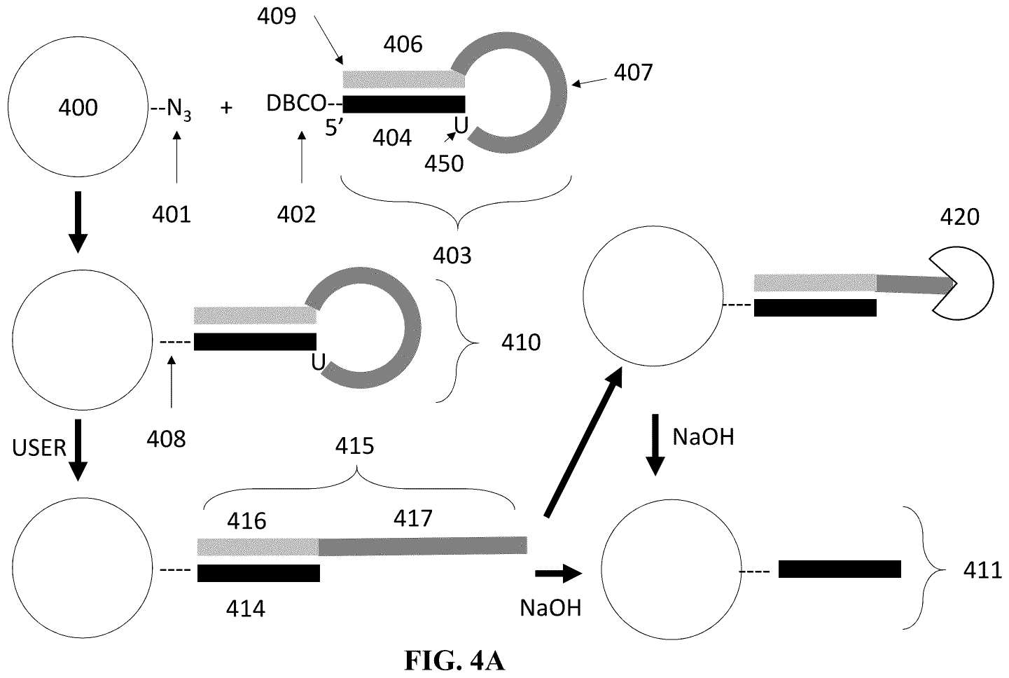

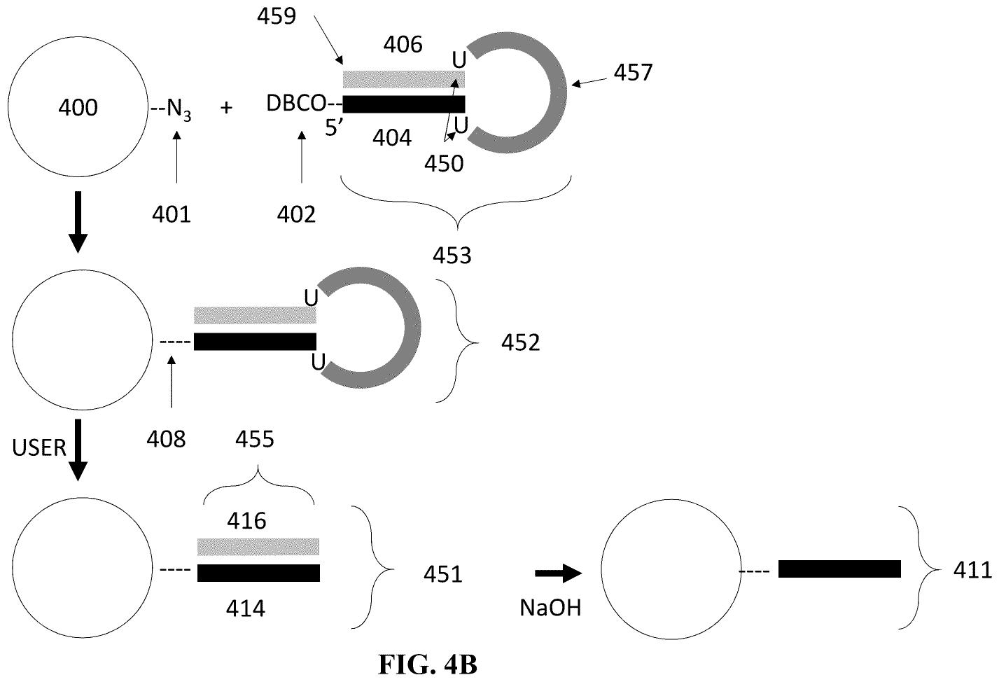

Reference is now made to FIG. 4A. FIG. 4A shows an alternative embodiment in which the bulky group is a hairpin. Support 400 comprises first coupling moiety 401, which is equivalent to support 100 and first coupling moiety 101 of FIG. 1A, and support 300 and first coupling moiety 301 of FIG. 3. Nucleic acid molecule 403 comprises only a single strand 409, which comprises a 5′ second coupling moiety 402. Single strand 409 comprises three regions: region 404, region 406 and region 407. Region 404 is equivalent to first strand 104 or 304 and will eventually be the single-stranded primer coupled to the support. Region 404 is the 5′ end of single strand 409 and comprises second coupling moiety 402 attached to its 5′ terminus. Region 406 is equivalent to regions 106 and 306 and is complementary and hybridized to region 404. Regions 404 and 406 are hybridized together to form a stalk of the hairpin. Region 407 is a single-stranded region that links regions 404 and 406. Region 407 is an unpaired loop of the hairpin. It will be understood by a skilled artisan that when second coupling moiety 402 is at the 5′ end of single strand 409, the order of the regions from 5′ to 3′ is region 404, region 407, and region 406. In an alternative embodiment, the second coupling moiety 402 may be at a 3′ end of single strand 409, and the regions in order from 5′ to 3′ are region 406, region 407, and region 404. In some embodiments, the first region comprises the second coupling moiety. In some embodiments, the second region comprises the second coupling moiety. In some embodiments, the first region is the most 5′ region of the strand. In some embodiments, the second region is the most 3′ region of the strand. In some embodiments, the second coupling moiety is at a 5′ terminus of the first region. In some embodiments, the first region is the most 3′ region of the strand. In some embodiments, the second region is the most 5′ region of the strand. In some embodiments, the second coupling moiety is at a 3′ terminus of the first region. In some embodiments, a terminus is the terminal nucleotide. In some embodiments, the single strand is 3′ blocked. In some embodiments, the terminal nucleotide of the single strand is a blocked nucleotide. In some embodiments, the second region is shorter than the first region. In some embodiments, the second region is complementary to only a portion of the first region and a portion of the first region is single-stranded. In some embodiments, the single-stranded portion is a 5′ portion. In some embodiments, the single-stranded portion comprises not more than 7, 8, 9, 10, 11, 12, 13, 14, 15, 16, 17, 18, 19, or 20 nucleotides. Each possibility represents a separate embodiment of the invention. In some embodiments, the single-stranded portion comprises not more than 10 nucleotides.

In some embodiments, the single strand is un-extendable. The terms “un-extendable”, “non-extendable” or “blocked” are interchangeable and may refer to the fact that a polynucleotide cannot be further polymerized by formation of phosphodiester bonds. In some embodiments, polymerization is template dependent or independent. In some embodiments, polymerization is enzyme dependent or independent. An un-extendable polynucleotide which can be used according to the method of the invention can be produced or comprise chemically modified nucleotides according to any method known in the art of molecular biology. In some embodiments, a 3′ hexanediol modified base renders a polynucleotide “un-extendable”. In some embodiments, dideoxynucleotide renders a polynucleotide “un-extendable”. In some embodiments, an un-extendable polynucleotide comprises a dideoxynucleotide. In some embodiments, an un-extendable polynucleotide comprises a 3′ hexanediol modified base. In some embodiments, the chemically modified nucleotides, e.g., a dideoxynucleotide or 3′ hexanediol modified base, is located at the 3′-end of the un-extendable polynucleotide.

In some embodiments, a single strand is 3′ blocked. As used herein, the term “3′ blocked” may also refer to a nucleotide that cannot be extended at its 3′ end by a polymerase. In some embodiments, a 3′ blocked strand comprises a 3′ modification or modified base. In some embodiments, the modification is a blocking modification. In some embodiments, the modified base is a blocked base. In some embodiments, a blocked base is a base to which polymerase cannot link a new base. In some embodiments, linking is polymerizing on a new base. In some embodiments, a blocked base is selected from a monophosphate nucleotide, a dideoxynucleotide and a 3′ hexanediol modified base. In some embodiments, a blocked base is a monophosphate nucleotide. In some embodiments, a blocked base is dideoxynucleotide. In some embodiments, a blocked base is a 3′ hexanediol modified base.

Region 407 can comprise a cleavable or excisable base 450. The cleavable or excisable base is shown here as a uracil (U) although any cleavable or excisable base may be employed. In some embodiments, the strand comprises at least one cleavable or excisable base. In some embodiments, the single-stranded region comprises at least one cleavable or excisable base. In some embodiments, the unhybridized region comprises at least one cleavable or excisable base. In some embodiments, loop comprises at least one cleavable or excisable base. In some embodiments, the cleavable or excisable base is proximal to the first region. In some embodiments, the cleavable or excisable base is proximal to the 3′ end of the first region. In some embodiments, the base adjacent to the first region is the cleavable or excisable base. In some embodiments, the cleavable or excisable base is within 5, 4, 3, 2, or 1 nucleotides of the first region. Each possibility represents a separate embodiment of the invention. In some embodiments, the cleavable or excisable base is within 5, 4, 3, 2, or 1 nucleotides of the 3′ end of the first region. Each possibility represents a separate embodiment of the invention.

As used herein, the term “cleavable or excisable base” generally refers to any base or analog of a base (e.g., nucleobase) that can be specifically cleaved and removed or excised from a nucleic acid molecule. The terms “cleavable” and “excisable” as used herein are synonymous and interchangeable. The terms “cleavage” and “excision” as used herein are synonymous and interchangeable. Examples of cleavable bases include, but are not limited to, uracil, 8-oxoguanine (also referred to as 8-hydroxyguanine, 8-oxo-7,8-dihydroguanine, 7,8-dihydro-8-oxoguanine, and 8oxoG herein), inosine, and 2,6-diamino-4-hydroxy-5-formamidopyrimidine (FapyG). In some embodiments, the uracil is a DNA uracil. In some embodiments, the uracil is an RNA uracil. Cleavage and/or excision of a cleavable or excisable moiety may be carried out by contacting the cleavable or excisable moiety (e.g., cleavable base) with a cleaving agent. Examples of cleaving agents include, but are not limited to, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), and RNase (e.g., RNaseH, such as RNaseHII). Photocleavable or photoexcisable moieties may be cleaved or excised using appropriate application of energy, such as by contacting the moiety with UV light. In some embodiments, a cleavable or excisable moiety is a cleavable or excisable base. One or more cleaving agents may be used in combination to cleave or excise a cleavable or excisable moiety. In some embodiments, the cleavable or excisable base is subjected to condition sufficient to cleave or excise it. In some embodiments, the suitable conditions comprise contacting the cleavable or excisable base with a cleaving agent configured to cleave or excise the at least one cleavable or excisable base. In an example, the cleavable base may be an RNA base in a DNA backbone, and the cleaving agent may be RNase (e.g., RNaseH or RNaseHII). In such a case, the nucleic acid molecule may not be an RNA molecule. In some embodiments, the cleavable or excisable base is an RNA base and the nucleic acid molecule s devoid of RNA bases other than the cleavable or excisable base. In another example, the cleavable base may be a uracil DNA base and the cleaving agent may be selected from uracil DNA glycosylase (UDG), apyrimidinic/apurinic endonuclease (APE), Endonuclease VIII and uracil-specific excision reagent (USER) enzyme. For example, the cleaving agent may be UDG. For example, the cleaving agent may be APE. In another example, the cleavable base may be an inosine base and the cleaving agent may be Endonuclease V (Endo V). In another example, the cleavable base may be 2,6-diamino-4-hydroxy-5-formamidopyrimidine (FapyG) base and the cleaving agent may be formamidopyrimidine DNA glycosylase (Fpg). In another example, the cleavable base may be 8-oxo-7,8-dihydroguanine (8oxoG) and the cleaving agent may be 8-oxoguanine glycosylase (OGG1). In another example, the cleavable base may be a photo-cleavable base and the cleaving agent may be light, such as laser light. Application of a cleaving agent may generate a “nick” in a strand of a nucleic acid molecule. Alternatively, or in addition to, another enzyme may be added to generate a nick, or otherwise functionalize a nick. For example, T4 PNK may be added to remove a 3′ phosphate. An enzyme may be used to remove a lesion, such as a 3′ lesion. In some embodiments, the cleavable or excisable base is an RNA base, and the cleaving agent is RNase H. In some embodiments, the RNase H is RNase HII. In some embodiments, the RNA base is a uracil RNA base. In some embodiments, the cleavable or excisable base is a uracil DNA base, and the cleaving agent is selected from a) UDG, b) UDG and an Endonuclease and c) USER. In some embodiments, the Endonuclease is Endonuclease VIII.

Coupling of first coupling moiety 401 to second coupling moiety 402 links nucleic acid molecule 403 to support 400 via bond 408. The result is coupled support 410. The bulk of the loop of the hairpin acts in much the same way as the mock-template portion or the polymer portion. This can be seen, for example, in FIG. 2C. It will be apparent that the size of the loop region is related to the space that will be taken up between the nucleic acid molecules. Thus, in some embodiments, the size of the loop may be inversely proportionate to the density of the nucleic acid molecules on the support. By altering the size of the loop, the density of the nucleic acid molecules on the support may be controlled. Additionally, the very presence of the second strand may also increase the space between nucleic acid molecules and may decrease density.

In some embodiments, the unhybridized region comprises at least 5, 6, 7, 8, 9, 10, 15, 20, 25, 30, 40, 50, 60, 70, 80, 90, 100, 120, 130, 140, 150, 200, 250, 300, 350, 400, 450, or 500 nucleotides. Each possibility represents a separate embodiment of the invention. In some embodiments, the unhybridized region comprises at least 7 nucleotides. In some embodiments, the unhybridized region comprises at least 20 nucleotides. In some embodiments, the unhybridized region comprises at most 50, 60, 70, 80, 90, 100, 150, 200, 250, 300, 350, 400, 450, 500, 550, 600, 650, 700, 750, 800, 850, 900, 950, or 1000 nucleotides. Each possibility represents a separate embodiment of the invention. In some embodiments, the unhybridized region comprises between 7-800, 7-750, 7-700, 7-650, 7-600, 7-550, 7-500, 10-800, 10-750, 10-700, 10-650, 10-600, 10-550, 10-500, 20-800, 20-750, 20-700, 20-650, 20-600, 20-550, 20-500, 50-800, 50-750, 50-700, 50-650, 50-600, 50-550, 50-500, 100-800, 100-750, 100-700, 100-650, 100-600, 100-550, 100-500, 200-800, 200-750, 200-700, 200-650, 200-600, 200-550, 200-500, 300-800, 300-750, 300-700, 300-650, 300-600, 300-550, 300-500, 400-800, 400-750, 400-700, 400-650, 400-600, 400-550, 400-500, 500-800, 500-750, 500-700, 500-650, 500-600, or 500-550 nucleotides. Each possibility represents a separate embodiment of the invention.

In some embodiments, at least a portion of the unhybridized region is unstructured. In some embodiments, the unhybridized region is a homopolymer. In some embodiments, the unhybridized region consists of a repeat of a single base and the at least one cleavable or excisable base. In some embodiments, the base is T. In some embodiments, the unhybridized region comprises a T homopolymer. In some embodiments, the unhybridized region consists of a T homopolymer and the at least one cleavable or excisable base. In some embodiments, the unhybridized region is not complementary to any other part of the nucleic acid molecule. In some embodiments, the homopolymer stretch comprises at least about 3, 5, 7, 9, or 10 bases. Each possibility represents the separate embodiment of the invention. In some embodiments, the homopolymer stretch comprises at least about 5 bases.

Coupled support 410 may be exposed to conditions sufficient to cleave or excise cleavable or excisable base 450. As in this example cleavable or excisable base 450 is a DNA uracil within a DNA background the condition is addition of USER enzyme although other conditions are possible, just as other cleavable or excisable bases are possible. This cleavage, being within the single-stranded region, produces two separate strands, fourth strand 414 and fifth strand 415. Strand 414 and strand 415 are hybridized to each other. Strand 414 is conjugated to the support. Strand 415 comprises two regions: region 416 which is hybridized to strand 414, and region 417 which is unhybridized. Region 417 is the same as region 407 but without cleavable or excisable base 450.

Denaturation of strand 415 from strand 414 may result in a support coupled to a single-stranded primer 411. Support coupled to a single-stranded primer 411 is the same conformation as supports 111 and 311. Coupled support 410 can be stored as is and only denatured just before subsequent enzymatic treatment. Alternatively, coupled support 410 can be treated with a 5′ to 3′ nuclease 420 that only digests single-stranded nucleic acids and not double-stranded. This will remove region 417, leaving region 416 hybridized to strand 415. This is parallel to the method demonstrated in FIG. 1B. Denaturation will still result in support coupled to a single-stranded primer 411. Denaturation is a beneficial step, as it prevents region 417 from acting as a template for a later enzymatic reaction.

In some embodiments, the method further comprises subjecting the coupled support to conditions sufficient to cleave or excise the at least one cleavable or excisable base. In some embodiments, suitable conditions comprise contacting the nucleic acid molecule with a cleaving agent configured to cleave or excise the at least one cleavable or excisable bases. In some embodiments, cleavage or excision of the cleavable or excisable base produces two separate strands. In some embodiments, the two separate strands are a fourth strand and a fifth strand. In some embodiments, the fourth strand comprises the second coupling moiety and is coupled to the solid support.

In some embodiments, the method further comprises contacting the nucleic acid molecule with a nuclease. In some embodiments, the method further comprises contacting the support coupled to the nucleic acid molecule with a nuclease. In some embodiments, the nuclease digests the unhybridized region of the fifth strand. In some embodiments, the method further comprises denaturing the fifth strand from the fourth strand. In some embodiments, the denaturing produces a solid support coupled to a plurality of single-stranded primers.

Reference is now made to FIG. 4B, which provides a variant of the method provided in FIG. 4A. In this embodiment, unhybridized region 457 comprises a plurality of cleavable or excisable bases 450 (shown here as uracil (U)). Single strand 459 of nucleic acid molecule 453 is still coupled to support 400 and this coupling produces coupled support 452. However, upon treatment with a cleaving agent (e.g., USER) all cleavable or excisable bases 450 are removed from region 457; this results in the complete removal of region 457 from the nucleic acid molecule (e.g., because there are cleavable or excisable bases at each end of region 457). The resultant fifth strand 455 consists of region 416 and the resultant coupled support 451 may comprise no single-stranded region or alternatively a very short one. Denaturation removes strand 416 from strand 414, resulting in a support coupled to a plurality of single-stranded primers 411. Once again, coupled support 451 can be used for storage and only denatured into coupled support 411 just before subsequent enzymatic reaction. This storage method results in less clumping of the capture supports in storage.

In some embodiments, the unhybridized region comprises a plurality of cleavable or excisable bases. In some embodiments, cleavage or excision of the plurality of cleavable or excisable bases removes at least a portion of the unhybridized region. In some embodiments, cleavage or excision of the plurality of cleavable or excisable bases removes the unhybridized region. In some embodiments, removal is removal from the nucleic acid molecule. In some embodiments, cleavage or excision of the plurality of cleavable or excisable bases degrades the unhybridized region. In some embodiments, at least one of the cleavable or excisable bases of the plurality of cleavable or excisable bases is proximal to terminus of the first region. In some embodiments, at least one of the cleavable or excisable bases of the plurality of cleavable or excisable bases is proximal to terminus of the second region. In some embodiments, at least one of the cleavable or excisable bases of the plurality of cleavable or excisable bases is proximal to the 3′ end of the first region. In some embodiments, at least one of the cleavable or excisable bases of the plurality of cleavable or excisable bases is proximal to the 5′ end of the second region. In some embodiments, at least one of the cleavable or excisable bases of the plurality of cleavable or excisable bases is adjacent to the 3′ end of the first region. In some embodiments, at least one of the cleavable or excisable bases of the plurality of cleavable or excisable bases is adjacent to the 5′ end of the second region. In some embodiments, a first cleavable or excisable base of the plurality of cleavable or excisable bases is adjacent to the 3′ end of the first region and a second cleavable or excisable base of the plurality of cleavable or excisable bases is adjacent to the 5′ end of the second region.

In some embodiments, the method further comprises contacting the coupled solid support to a target nucleic acid molecule. In some embodiments, the target nucleic acid molecule is a molecule of a library. In some embodiments, the target nucleic acid molecule comprises a target sequence. In some embodiments, the target sequence is complementary to the single-stranded primers. In some embodiments, complementary is reverse complementary. In some embodiments, the target sequence is a 3′ region of the target nucleic acid molecule. In some embodiments, the 3′ region is a 3′ terminal region. In some embodiments, the target sequence is a 5′ region of the target nucleic acid molecule. In some embodiments, the 5′ region is a 5′ terminal region. In some embodiments, the contacting is under condition sufficient to hybridize the target nucleic acid molecule to the single-stranded nucleic acid primer. In some embodiments, the contacting is performed under conditions sufficient to hybridize the target sequence to the single-stranded nucleic acid primer. In some embodiments, hybridizing produces a solid support coupled to the target nucleic acid molecule. In some embodiments, hybridizing produces a solid support coupled target nucleic acid molecule. In some embodiments, the contacting comprises annealing the target nucleic acid molecules to the single-stranded primers. In some embodiments, a single target nucleic acid molecule is annealed to a single support. In some embodiments, multiple target nucleic acid molecules are annealed to a single support. In some embodiments, a single target nucleic acid molecule is annealed to each support of a plurality of supports. In some embodiments, annealing produces a solid support coupled target nucleic acid molecule.

In some embodiments, the method further comprises extending the single-stranded nucleic acid primer to produce a nucleic acid strand that is complementary to the target nucleic acid molecule. In some embodiments, complementary is reverse complementary. In some embodiments, the produced nucleic acid strand is coupled to the solid support. In some embodiments, the produced nucleic acid strand is directly coupled to the solid support and not merely connected via hybridization. In some embodiments, directly coupled is covalently linked. In some embodiments, the primer is extended from a 3′ end. In some embodiments, the extending comprises contacting the primer with a polymerase. In some embodiments, the extending comprises adding reagents sufficient for extending. In some embodiments, the extending comprises amplification. In some embodiments, amplification comprises contacting the primer with a polymerase. In some embodiments, the polymerase is a DNA polymerase. In some embodiments, the polymerase is an RNA polymerase. In some embodiments, the amplification comprises adding reagents sufficient for amplification. In some embodiments, the extending comprises adding reagents sufficient for extension. In some embodiments, the reagents comprise free nucleotides.

In some embodiments, the method further comprises amplifying the target nucleic acid molecule. In some embodiments, the amplifying comprises using the single-stranded primers coupled to the solid support. In some embodiments, the amplification is by a polymerase chain reaction (PCR). In some embodiments, the amplification is clonal amplification.

The conditions encountered during the annealing steps of a PCR reaction will be generally known to one skilled in the art, although the precise annealing conditions will vary from reaction to reaction (for example, see Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.). Typically, such conditions may comprise, but are not limited to, a denaturing step at a temperature of about 94° C. for about one minute, followed by exposure to a temperature in the range of from 40° C. to 72° C. (preferably 50-68° C.) for a period of about 1 minute in standard PCR reaction buffer.

Different annealing conditions may be used for a single primer extension reaction not forming part of a PCR reaction (for example, see Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.). Conditions for primer annealing in a single primer extension include, for example, exposure to a temperature in the range of from 30 to 37° C. in standard primer extension buffer. It will be appreciated that different enzymes, and hence different reaction buffers, may be used for a single primer extension reaction as opposed to a PCR reaction. There is no requirement to use a thermostable polymerase for a single primer extension reaction.

The term “annealing” as used in this context refers to sequence-specific binding/hybridization of a primer, e.g., a single-stranded primer of a capture substrate, to a primer-binding sequence in an adapter region of an adapter-target construct, e.g., a target nucleic acid molecule, under the conditions to be used for the primer annealing step of the initial primer extension reaction.

The products of the primer extension reaction may be subjected to standard denaturing conditions in order to separate the extension products from strands of the adapter-target constructs. Optionally, the strands of the adapter-target constructs may be removed at this stage. The extension products (with or without the original strands of the adapter-target constructs) collectively form a library of template polynucleotides which can be used as templates for PCR.

If desired, the initial primer extension reaction may be repeated one or more times, through rounds of primer annealing, extension, and denaturation, in order to form multiple copies of the same extension products complementary to the adapter-target constructs.

The products of further PCR amplification may be collected to form a library of templates comprising “amplification products derived from” the initial primer extension products. In some embodiments, both primers used for further PCR amplification will anneal to different primer-binding sequences on opposite strands in the overhang region of the first adapter and the primer/second adapter. Other embodiments may, however, be based on the use of a single type of amplification primer which anneals to a primer-binding sequence in the double-stranded region of the adapter. In embodiments of the method based on PCR amplification the “initial” primer extension reaction occurs in the first cycle of PCR.

Inclusion of the initial primer extension step (and optionally further rounds of PCR amplification) to form complementary copies of the adapter-target constructs (prior to whole genome or solid-phase PCR) may be advantageous, for several reasons. Firstly, inclusion of the primer extension step, and subsequent PCR amplification, acts as an enrichment step to select for adapter-target constructs with adapters ligated at both ends. Only target constructs with adapters ligated at both ends provide effective templates for whole genome or solid-phase PCR using common or universal primers specific for primer-binding sequences in the adapters, hence it is advantageous to produce a template library comprising only double-ligated targets prior to solid-phase or whole genome amplification.

In some embodiments, the PCR performed is emulsion PCR. In some embodiments, clonal copies of the adapter target constructs, or complementary copies thereof, are produced on solid support using emulsion PCR. Methods of performing emulsion PCR and producing clonal copies on solid supports can be found in U.S. Pat. No. 8,765,380 and International Patent Application WO2019079653, the contents of which are herein incorporated by reference. Methods of performing sequencing by synthesis on clonal populations can be found in at least U.S. Pat. Nos. 9,902,951 and 8,772,473, the contents of which are herein incorporated by reference.

Capture Substrates

By another aspect, there is provided a composition comprising a plurality of solid supports coupled to nucleic acid molecules produced by a method of the invention.

In some embodiments, the composition is a capture substrate. In some embodiments, the composition is for use in capturing a target nucleic acid molecule. In some embodiments, the target nucleic acid molecules are template nucleic acid molecules. In some embodiments, the nucleic acid molecules are single-stranded. In some embodiments, the nucleic acid molecules are single-stranded primers. In some embodiments, the nucleic acid molecules are double-stranded. In some embodiments, the double-stranded molecules can be denatured to produce single-stranded primers.

By another aspect, there is provided a capture substrate, comprising a support coupled to a plurality of primers, wherein each primer is hybridized to a complementary nucleic acid molecule and wherein each complementary nucleic acid molecule is devoid of an unhybridized region of greater than 10 nucleotides.

By another aspect, there is provided a capture substrate comprising a support comprising first coupling moieties and wherein a first portion of the first coupling moieties are coupled to a nucleic acid molecule and a second portion of the first coupling moieties are not coupled to a nucleic acid molecule and wherein less than half of all first coupling moieties are coupled to a nucleic acid molecule.

By another aspect, there is provided a capture substrate comprising a support comprising first coupling moieties and wherein a first portion of the first coupling moieties are coupled to a nucleic acid molecule and a second portion of the first coupling moieties are not coupled to a nucleic acid molecule and wherein the nucleic acid molecules are present at less than a predetermined percentage of a maximal density.

By another aspect, there is provided a capture substrate comprising a support comprising between about 5,000 and about 75,000 nucleic acid molecules coupled thereto.

By another aspect, there is provided a capture substrate comprising at least two first coupling moieties coupled to a nucleic acid molecule and wherein the distance between a first coupling moiety coupled to a nucleic acid molecule and an adjacent first coupling moiety coupled to a nucleic acid molecule is at least about 2 nm.

In some embodiments, each complementary nucleic acid molecule is devoid of a 5′ unhybridized region. In some embodiments, each complementary nucleic acid molecule is devoid of a 3′ unhybridized region. In some embodiments, an unhybridized region is an overhang. In some embodiments, each complementary nucleic acid molecule is devoid of an unhybridized region of greater than 10, 9, 8, 7, 6, 5, 4, 3, 2, or 1 nucleotide. Each possibility represents a separate embodiment of the invention. In some embodiments, each complementary nucleic acid molecule is devoid of an unhybridized region of greater than 5 nucleotides. In some embodiments, each complementary nucleic acid molecule is devoid of an unhybridized region of greater than 1 nucleotide. In some embodiments, each complementary nucleic acid molecule is devoid of an unhybridized region of any length. In some embodiments, the complementary nucleic acid molecule consists of a double-stranded region. In some embodiments, the complementary nucleic acid molecule is devoid of a single-stranded region.

In some embodiments, the nucleic acid molecule is a double-stranded nucleic acid molecule and the strand not coupled to the support is devoid of an unhybridized region. In some embodiments, the unhybridized region is a 3′ unhybridized region. In some embodiments, the unhybridized region is a 5′ unhybridized region. In some embodiments, the unhybridized region is a single-stranded region. In some embodiments, the unhybridized region is an unhybridized region of greater than 10, 9, 8, 7, 6, 5, 4, 3, 2, or 1 nucleotide. Each possibility represents a separate embodiment of the invention. In some embodiments, the unhybridized region is an unhybridized region of greater than 7 nucleotides. In some embodiments, the unhybridized region is an unhybridized region of greater than 5 nucleotides. In some embodiments, the unhybridized region is an unhybridized region of greater than 1 nucleotide. In some embodiments, the unhybridized region is any unhybridized region of any length.

In some embodiments, the primer and complementary nucleic acid molecule are two separate strands. In some embodiments, the primer and complementary nucleic acid molecule are a double-stranded molecule. In some embodiments, the primer and nucleic acid molecule are not part of a single strand. In some embodiments, the complementary nucleic acid molecule is devoid of a tag. In some embodiments, a tag is a detectable moiety. In some embodiments, a tag is a label. In some embodiments, a detectable moiety is a fluorescent moiety.

In some embodiments, the complementary nucleic acid molecule is devoid of a capture entity. In some embodiments, a capture entity is a capture moiety. In some instances, the capture entity may comprise biotin (B), such that the primer molecule is biotinylated. In some instances, the capture entity may comprise a capture sequence (e.g., a nucleic acid sequence). In some instances, a sequence of the primer molecule may function as a capture sequence. In other instances, the capture entity may comprise another nucleic acid molecule comprising a capture sequence. In some instances, the capture entity may comprise a magnetic particle capable of capture by application of a magnetic field. In some instances, the capture entity may comprise a charged particle capable of capture by application of an electric field. In some instances, the capture entity may comprise one or more other mechanisms configured for, or capable of, capture by a capturing molecule. As used herein, a “capture entity” is a molecule that can be isolated by binding to a capturing molecule. For example, the oligonucleotide can be conjugated to biotin (capture entity) and then captured by a streptavidin column (the capturing molecule).

In some embodiments, the support comprises at least about 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000 or 300,000 first coupling moieties. Each possibility represents a separate embodiment of the invention. In some embodiments, the support comprises at least about 150,000 first coupling moieties. In some embodiments, the support comprises a density of at least about 10,000, 20,000, 30,000, 40,000, 50,000, 60,000, 70,000, 80,000, 90,000, or 100,000 first coupling moieties per cubic micron. Each possibility represents a separate embodiment of the invention. In some embodiments, the support comprises a density of at least about 50,000 first coupling moieties per cubic micron.

In some embodiments, each solid support of the population is coupled to nucleic acid molecules at less than a maximal density. The maximal density being the density achieved when only the first strand alone is coupled to the support. In some embodiments, the maximal density comprises all first coupling moieties coupled to a nucleic acid molecule. In some embodiments, the density is the percent occupancy of the first coupling moieties by nucleic acid molecules. In some embodiments, the maximal density comprises the maximum number of first coupling moieties on a given support that can couple to any nucleic acid molecule being coupled. In some embodiments, the maximal density comprises the maximum number of first coupling moieties on a given support that can couple to any nucleic acid molecule of the same length as the first strand being coupled. It will be understood that the maximum number of bound molecules will depend on the molecule being bound. For short single-stranded molecules, the maximum number that can be bound is essentially equal to the number of first coupling moieties. In some embodiments, short is equal to or less than about 15, 20, 25, 30, 35, 40, or 45 nucleotides in length. Each possibility represents a separate embodiment of the invention. In some embodiments, short is equal to or less than about 15 nucleotides in length. In some embodiments, short is equal to or less than about 45 nucleotides in length.

In some embodiments, less than maximal density is a predetermined percentage of maximal density. In some embodiments, less than maximal density is less than a predetermined percentage of maximal density. In some embodiments, the predetermined percentage is about 20%, about 25%, about 30%, about 40%, about 50%, about 60%, about 70%, about 75%, about 80%, or about 90%. Each possibility represents a separate embodiment of the invention. In some embodiments, the predetermined percentage is about 50%. In some embodiments, less than 50% of maximal density comprises at least half of said first coupling moieties not coupled to a nucleic acid molecule.

In some embodiments, each support is coupled to not more than about 80,000, 75,000, 70,000, 65,000, 60,000, 55,000, 50,000, 45,000, 40,000, 35,000, 30,000, 25,000, 20,000, 15,000, or 10,000 nucleic acid molecules. Each possibility represents a separate embodiment of the invention. In some embodiments, each support is coupled to not more than about 75,000 nucleic acid molecules. In some embodiments, each support is coupled to not more than about 50,000 nucleic acid molecules. In some embodiments, each support is coupled to not more than 10,000 nucleic acid molecules. In some embodiments, not more than is less than. In some embodiments, the threshold number is about 80,000, 75,000, 70,000, 65,000, 60,000, 55,000, 50,000, 45,000, 40,000, 35,000, 30,000, 25,000, 20,000, 15,000, or 10,000 nucleic acid molecules. Each possibility represents a separate embodiment of the invention. In some embodiments, the threshold number is about 75,000. In some embodiments, the threshold number is 50,000. In some embodiments, the threshold number is about 10,000. In some embodiments, the threshold number is less than the maximum. In some embodiments, the maximum is the maximum number of nucleic acid molecules that can be coupled to the solid support. In some embodiments, the maximum is the maximum number of nucleic acid molecules of the same length as the first strand that can be coupled to the solid support.

In some embodiments, each support is coupled to nucleic acid molecules at a density of not more than about 170,000, 165,000, 160,000, 155,000, 150,000, 145,000, 140,000, 135,000, 130,000, 125,000, 120,000, 115,000, 110,000, 105,000, 100,000, 95,000, 90,000, 85,000, 80,000, 75,000, 70,000, 65,000, 60,000, 55,000, 50,000, 45,000, 40,000, 35,000, 30,000, 25,000, 20,000, 15,000, 10,000, 7,500, 5,000 or 2,500 nucleic acid molecules per cubic micron. Each possibility represents a separate embodiment of the invention. In some embodiments, each support is coupled to not more than about 170,000 nucleic acid molecules. In some embodiments, each support is coupled to not more than about 150,000 nucleic acid molecules. In some embodiments, each support is coupled to not more than about 75,000 nucleic acid molecules. In some embodiments, each support is coupled to not more than about 50,000 nucleic acid molecules. In some embodiments, each support is coupled to not more than about 10,000 nucleic acid molecules. In some embodiments, not more than is less than. In some embodiments, each solid support is coupled to between about 5,000-75,000 nucleic acid molecules. In some embodiments, each solid support is coupled to between about 5,000-10,000 nucleic acid molecules.

In some embodiments, the nucleic acid molecules are single-stranded. In some embodiments, the nucleic acid molecules are double-stranded. In some embodiments, the nucleic acid molecules are primers. In some embodiments, the nucleic acid molecules are devoid of single-stranded regions. In some embodiments, the nucleic acid molecules are devoid of single-stranded overhangs. In some embodiments, the nucleic acid molecules comprise a length of not more than about 45 nucleotides. In some embodiments, the nucleic acid molecules comprise a length of at least about 15 nucleotides.

In some embodiments, the beads comprise a diameter of greater than about 0.1 μm, 0.2 μm, 0.3 μm, 0.4 μm, 0.5 μm, 0.6 μm, 0.7 μm, 0.8 μm, 0.9 μm, or 1 μm. Each possibility represents a separate embodiment of the invention. In some embodiments, the beads comprise a diameter of greater than about 0.2 μm. In some embodiments, the beads comprise a diameter of greater than about 0.5 μm. In some embodiments, the beads comprise a diameter of less than about 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. Each possibility represents a separate embodiment of the invention. In some embodiments, the beads comprise a diameter of less than about 2.5 μm. In some embodiments, the beads comprise a diameter of between about 0.1-5 μm, 0.1-4 μm, 0.1-3 μm, 0.1-2.5 μm, 0.1-2 μm, 0.1-1.5 μm, 0.1-1 μm, 0.2-5 μm, 0.2-4 μm, 0.2-3 μm, 0.2-2.5 μm, 0.2-2 μm, 0.2-1.5 μm, 0.2-1 μm, 0.3-5 μm, 0.3-4 μm, 0.3-3 μm, 0.3-2.5 μm, 0.3-2 μm, 0.3-1.5 μm, 0.3-1 μm, 0.4-5 μm, 0.4-4 μm, 0.4-3 μm, 0.4-2.5 μm, 0.4-2 μm, 0.4-1.5 μm, 0.4-1 μm, 0.5-5 μm, 0.5-4 μm, 0.5-3 μm, 0.5-2.5 μm, 0.5-2 μm, 0.5-1.5 μm, or 0.5-1 μm. Each possibility represents a separate embodiment of the invention. In some embodiments, the beads comprise a diameter of between about 0.2-2.5 μm. In some embodiments, the beads comprise a diameter of between about 0.5-2.5 μm.

In some embodiments, the coupled solid support comprises first coupling moieties coupled to the second coupling moiety and first coupling moieties not coupled to the second coupling moiety. In some embodiments, the distance between adjacent first coupling moieties coupled to the second coupling moiety is at least about 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, or 20 nm. Each possibility represents a separate embodiment of the invention. In some embodiments, the distance is the average distance. In some embodiments, average distance is average distance on the support. In some embodiments, the distance between adjacent first coupling moieties coupled to the second coupling moiety is at least about 2 nm. In some embodiments, the distance between adjacent first coupling moieties coupled to the second coupling moiety is at least about 10 nm. In some embodiments, the coupled solid support comprises at least one first coupling moiety coupled to a second coupling moiety that is at least about 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, 15 nm, 16 nm, 17 nm, 18 nm, 19 nm, or 20 nm distant from another first coupling moiety coupled to a second coupling moiety. Each possibility represents a separate embodiment of the invention. In some embodiments, the coupled solid support comprises at least one first coupling moiety coupled to a second coupling moiety that is at least about 2 nm. In some embodiments, the coupled solid support comprises at least one first coupling moiety coupled to a second coupling moiety that is at least about 10 nm.

In some embodiments, the average distance between a first first coupling moiety and second first coupling moiety is less than about 0.1 nm, 0.2 nm, 0.3 nm, 0.4 nm, 0.5 nm, 0.6 nm, 0.7 nm, 0.8 nm, 0.9 nm, 1 nm, 1.1 nm, 1.2 nm, 1.3 nm, 1.4 nm, 1.5 nm, 1.6 nm, 1.7 nm, 1.8 nm, 1.9 nm, 2 nm, 2.1 nm, 2.2 nm, 2.3 nm, 2.4 nm, 2.5 nm, 2.6 nm, 2.7 nm, 2.8 nm, 2.9 nm, or 3 nm. Each possibility represents a separate embodiment of the invention. In some embodiments, the average distance between a first first coupling moiety and second first coupling moiety is less than about 2 nm. In some embodiments, the average distance between a first first coupling moiety and second first coupling moiety is less than about 1 nm.

In some embodiments, the support comprises at least about 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000 or 300,000 first coupling moieties. Each possibility represents a separate embodiment of the invention. In some embodiments, the support comprises at least about 150,000 first coupling moieties. In some embodiments, the support comprises at least about 200,000 first coupling moieties.

In some embodiments, the solid support is coupled to the nucleic acid molecule by a bond. In some embodiments, the bond is a covalent bond. In some embodiments, the bond is not hybridization. In some embodiments, the bond is not non-specific binding. In some embodiments, the bond is a bond produced via a click reaction. In some embodiments, a click reaction is a reaction using click chemistry. In some embodiments, the support is coupled to a 5′ end of the nucleic acid molecule. In some embodiments, the support is coupled to a 3′ end of the nucleic acid molecule. In some embodiments, an end is a terminal nucleotide.

By another aspect, there is provided a composition comprising the capture substrate of the invention.

In some embodiments, the composition is a storage solution. In some embodiments, the solution is a solution for use in capturing a target nucleic acid molecule. In some embodiments, the capture substrate is present in the solution at a density sufficient for hybridization between single-stranded nucleic acid molecule of different capture substrates. In some embodiments, the capture substrate is a capture substrate comprising a double-stranded nucleic acid molecule. In some embodiments, the double-stranded molecule does not comprise an unhybridized region. In some embodiments, the double-stranded molecule does not comprise an overhang. In some embodiments, the double-stranded molecule does not comprise a single-stranded region.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

A support with reduced density of bound nucleic acid molecules was generated in according with the method provided in FIG. 1A. Beads with a crosslinked polystyrene core and a polyacrylamide coated surface were functionalized with azide. A first strand DNA oligonucleotide was generated with the following sequence: 5′ CCTATCCCCTGTGTGCCTTGGCAGTCTC 3′ (SEQ ID NO: 1). It was coupled at its 5′ end to a DBCO molecule. A second strand was generated with an irrelevant sequence of ˜400 nucleotides in length at the 5′ end and a 3′ terminal sequence of 5′ GAGACTGCCAAGGCACACAGGGGATAGG 3′ (SEQ ID NO: 2). This length was selected as it is similar to the average sized produced during standard library prep and thus would be roughly equivalent to the target molecules to be bound and amplified. This 3′ terminal sequence is the reverse complement of the first strand and results in perfect hybridization. The two strands were mixed in an annealing buffer comprising 10 mM Tris, 100 mM NaCl, and 0.05% Triton X-100, and hybridized in a thermocycler by heating to 95° C. and cooling slowly to 25° C. A slight excess of second strand was used to ensure that all first strands were hybridized. The resultant double-stranded molecules were purified. The beads were then reacted with either the first strand alone or with the double-stranded molecule. An excess of first strand and double-stranded molecules was added to the beads (˜40 μM nucleic acids added to ˜10,000,000 beads/μL). The click reaction of azide and DBCO produced a covalent bond linking the first strand to the beads, and the reaction was allowed to proceed to completion such that all nucleic acids that could bind were able to.

To test how many nucleic acid molecules (either just the first strand or the double-stranded molecule) had coupled to the beads, the beads with double-stranded molecules were treated with NaOH to denature the second strands, which were then removed. Specifically, the beads were placed in 100 mM NaOH+0.05% Triton X-100 and vortexed. The beads were pelleted by centrifugation, the supernatant removed, and the wash repeated. Two more washes were performed with TET buffer (10 mM Tris+0.05% Triton X-100+1 mM EDTA), and the beads were then resuspended in TET buffer. Next, dye-labeled (FAM) oligonucleotides with reverse complementary to the first strand were added and allowed to hybridize to first strands present on the beads. Fluorescence was measured with a standard FACS machine and is proportionate to the number of first primers coupled to each bead (i.e., as indicated by the FAM nucleotides hybridized to first strands). The mean FL1-A fluorescence from the beads exposed to only the first primer was 26,272 units, whereas the mean fluorescence from the beads exposed to the double-stranded molecules was only 12,693 units. Background fluorescence from naked beads mixed with the labeled oligonucleotide was negligible. Thus, if the maximum binding possible is considered to be that achieved with the single-stranded primer, the double-stranded molecule produced binding of only 48% of the maximum. This confirms that use of a longer second strand as a bulky group is sufficient to space the nucleic acid molecules that can bind to the support and effectively lower the density of primers.

This experiment was repeated using a second strand with no overhang. That is, a strand that is the reverse complement of the first strand without any additional nucleotides. This very short strand produced binding of only 84% of the maximum. This second experiments confirms that by altering the length of the second strand, and thereby its bulkiness, one can control the density of binding. Lengthening the strand produces reduced density of primers on the beads, while shortening the strand will increase the density of primers on the beads.

The same experiment was repeated with the variation shown in FIG. 1B. Following coupling of the double-stranded molecules, the coupled beads were treated with Mung Bean Nuclease. 10 million beads per μL were mixed with 20 units MBN per billion beads in 1×MBN reaction buffer (NEB). The reaction was incubated at 30° C. for 30 minutes, after which it was inactivated with SDS. The beads were washed in TET buffer, followed by denaturation and removal of the second strand as described before. FACS analysis showed 49% of maximal coupling, nearly identical to the measurement recorded without blunting of the second strand.

The variation shown in FIG. 3 was also tested. The same first strand was used, but the second strand contained only the reverse complement of the first strands with a 5′ DBCO. An azide functionalized PEG20K polymer was reacted to produce a copolymer comprising PEG20K covalently bound to the 5′ end of SEQ ID NO: 2. The copolymer second strand was hybridized to the first strand (a slight excess of second strand was used) and then the resultant double-stranded molecule was purified. The beads were then reacted with either the first strand alone or with the double-stranded molecule. An excess of first strand and double-stranded molecules were added to the beads. The click reaction of azide and DBCO produced a covalent bond linking the first strand to the beads and the reaction was allowed to proceed to completion such that all nucleic acids that could bind were able to. Following denaturation, the occupancy was measured as before using FACS and, similar to the other methods, 49% of the maximal density was observed when the copolymer molecule was used.

The method shown in FIG. 4A was also tested. A hairpin molecule was generated with a 5′ linked DBCO molecule and the sequence 5′ CCTATCCCCTGTGTGCCTTGGCAGTCTCUTTTTTTTGACTGCCAAGGC 3′ (SEQ ID NO: 3). A uracil DNA base was used as the cleavable or excisable base and the C at the 3′ end was a dideoxycytosine. Following the coupling reaction, the coupled beads were treated with USER according to the manufacturer's protocol and the newly made second strand was denatured from the primer. The occupancy was measured as before using FACS, however, this molecule did not result in a significant reduction in density of bound primers. This may be due to the short single-stranded loop used which did not provide sufficient bulk to occlude azides on the beads.

In order to test that the reduction in primers on the beads did not hinder amplification, beads with primers produces by the first method (a mock-template second strand) and beads produce with addition of only single-stranded primers (the standard method) were used for emPCR clonal amplification. Libraries generated either from the human or E. coli genome were used for amplification. After amplification, quantification was performed by hybridizing an Atto dye-labeled probe sequence to a universal sequence present at the distal end of the bead-extended amplicon. The mean FL4 was roughly equivalent between the two types of beads, and indeed the bead produced with the lower density of primers actually showed a slight increase in FL4-A fluorescence though this was not statistically significant. When this fluorescence from amplification was compared to FL1-A fluorescence used to measure the number of primers on the bead (an aliquot of beads was removed before amplification and tested for primer density with a labeled probe), it was found that the beads produced with a high density of single-stranded primers had very low occupancy by amplified copies. Indeed, less than 15% of the primers coupled to the beads were being used as a primer for amplification and thus were occupied by fluorescent DNA strands. In contrast, the beads produced with a lower density of single-stranded primers had a much greater usage percentage with greater than 40% of the primers occupied with fluorescent DNA strands. This result demonstrates that the reduction in primer density does not remove essential primers that would be used for amplification, but rather removes excess primers that are wasted as they will never be used to produce an amplified copy of template. The new beads with a lower density of primers are therefore superior to the old beads and result in far less waste of expensive oligonucleotides. This is in addition to the benefit of reduced clumping/aggregation that occurs when there are no single-stranded molecules present that can produce inter-bead hybridization.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method of controlling density of nucleic acid molecules coupled to solid supports, the method comprising: a. providing a plurality of solid supports, wherein each solid support comprises a plurality of first coupling moieties; and b. contacting the plurality of solid supports with a plurality of nucleic acid molecules under conditions sufficient to couple the first coupling moiety to a second coupling moiety of the plurality of nucleic acid molecules to produce a coupled solid support, wherein each nucleic acid molecule comprises either: i. a first strand and a second strand, wherein the first strand comprises a 5′ linked second coupling moiety configured to couple to the first coupling moiety, or ii. a third strand comprising a double-stranded region in which a first region of the third strand is hybridized to a second region of the third strand, and wherein the third strand comprises a 5′ linked second coupling moiety configured to couple to the first coupling moiety; thereby producing a population of coupled solid supports that are coupled to nucleic acid molecules, wherein each coupled solid support is coupled to the nucleic acid molecules at a density that is less than a predetermined percentage of a maximal density. 2. The method of embodiment 1, wherein the maximal density comprises all of the plurality of first coupling moieties of the solid support being coupled to a nucleic acid molecule. 3. The method of embodiment 1 or 2, wherein the predetermined percentage is about 50%, about 60%, about 70%, about 80%, or about 90%. 4. The method of any one of embodiments 1 to 3, wherein the density that each coupled solid support is coupled to nucleic acid molecules is less than 75,000 nucleic acid molecules per cubic micron. 5. The method of embodiment 4, wherein the density that each coupled solid support is coupled to nucleic acid molecules is less than 20,000 nucleic acid molecules per square micron.

6. A method of controlling a number of nucleic acid molecules coupled to solid supports, the method comprising: a. providing a plurality of solid supports, wherein each solid support comprises a plurality of first coupling moieties; and b. contacting the plurality of solid supports with a plurality of nucleic acid molecules under conditions sufficient to couple the first coupling moiety to a second coupling moiety of the plurality of nucleic acid molecules to produce a coupled solid support, wherein each nucleic acid molecule comprises either: i. a first strand and a second strand, wherein the first strand comprises a 5′ linked second coupling moiety configured to couple to the first coupling moiety, or ii. a third strand comprising a double-stranded region in which a first region of the third strand is hybridized to a second region of the third strand, and wherein the third strand comprises a 5′ linked second coupling moiety configured to couple to the first coupling moiety; thereby producing a population of coupled solid supports that are coupled to nucleic acid molecules, wherein each coupled solid support is coupled to less than a threshold number of nucleic acid molecules. 7. The method of embodiment 6, wherein the threshold number of nucleic acid molecules is about 75,000 nucleic acid molecules. 8. The method of embodiment 6, wherein the threshold number of nucleic acid molecules is about 10,000 nucleic acid molecules. 9. The method of embodiment 6 or 7, wherein the threshold number of nucleic acid molecules is less than a maximum number of nucleic acid molecules that can be coupled to the solid support.

10. The method of any one of embodiments 1 to 9, wherein each nucleic acid molecule comprises (1) the first strand and the second strand, and (2) a double-stranded region in which at least a portion of the first strand is hybridized to at least a portion of the second strand. 11. The method of any one of embodiments 1 to 10, wherein the first coupling moiety does not comprise a nucleic acid. 12. The method of any one of embodiments 1 to 11, further comprising: (c) denaturing the second strand from the first strand to produce a coupled solid support that is coupled to a plurality of single-stranded primers. 13. The method of any one of embodiments 1 to 12, wherein the solid support is a bead. 14. The method of embodiment 13, wherein the bead is a microbead. 15. The method of embodiment 14, wherein the microbead has a diameter of between about 0.2 and about 2.5 microns. 16. The method of any one of embodiments 1 to 15, wherein the first coupling moiety and the second coupling moiety couple by forming a covalent bond. 17. The method of embodiment 16, wherein the first coupling moiety and the second coupling moiety couple by click chemistry. 18. The method of embodiment 17, wherein the coupling moiety is an azide molecule and the second coupling moiety is a diarylcyclooctyne moiety, or wherein the first coupling moiety is a diarylcyclooctyne moiety and the second coupling moiety is an azide molecule. 19. The method of embodiment 18, wherein the diarylcyclooctyne moiety is dibenzylcyclooctyne (DBCO). 20. The method of any one of embodiments 1 to 19, wherein the first strand comprises 15 to 45 nucleotides. 21. The method of any one of embodiments 1 to 20, wherein the second strand comprises 15 to 650 nucleotides. 22. The method of embodiment 21, further comprising: contacting the coupled solid support with a 5′ to 3′ nuclease capable of degrading a 5′ unhybridized region of the second strand under conditions sufficient to degrade the 5′ unhybridized region of the second strand and leave a hybridized region of the second strand hybridized to the first strand. 23. The method of any one of embodiments 1 to 20, wherein the second strand of the nucleic acid molecules is linked at a 5′ terminus to a non-nucleotide polymer. 24. The method of embodiment 23, wherein the second strand is linked to the non-nucleotide polymer by a covalent bond. 25. The method of embodiment 23 or 24, wherein the second strand is linked to the non-nucleotide polymer by click chemistry. 26. The method of any one of embodiments 23 to 25, wherein the non-nucleotide polymer is a synthetic polymer. 27. The method of any one of embodiments 23 to 26, wherein the non-nucleotide polymer has a size of between about 10,000 g/mol to about 700,000 g/mol. 28. The method of any one of embodiments 23 to 27, wherein the non-nucleotide polymer has a radius of between about 1 nm and about 100 nm. 29. The method of any one of embodiments 23 to 28, wherein the non-nucleotide polymer is selected from the group consisting of: a polyethylene glycol (PEG) polymer, a poly-L-glutamate (poly(L-glu)) polymer, and a combination thereof. 30. The method of any one of embodiments 23 to 29, further comprising, after denaturing the second strand from the first strand, collecting the second strands after the denaturing, and reusing them in the method of any one of embodiments 23 to 29. 31. The method of any one of embodiments 1 to 20, wherein the first region of the third strand is a 5′ region of the third strand and the second region of the third strand is a 3′ region of the third strand. 32. The method of embodiment 31, wherein the third strand further comprises an unhybridized region between the first region and the second region and wherein the unhybridized region comprises at least one cleavable or excisable base. 33. The method of embodiment 32, further comprising subjecting the coupled solid support to conditions sufficient to cleave or excise the at least one cleavable or excisable base to produce two separate strands. 34. The method of embodiment 33, wherein the conditions comprise contacting the nucleic acid molecule with a cleaving agent configured to cleave or excise the at least one cleavable or excisable base. 35. The method of embodiment 34, wherein the cleaving agent is selected from the group consisting of: uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and any combination thereof. 36. The method of any one of embodiments 32 to 35, wherein the at least one cleavable or excisable base is selected from the group consisting of: a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, a photocleavable base, and any combination thereof. 37. The method of any one of embodiments 32 to 36, wherein the at least one cleavable or excisable base is adjacent to a 3′ end of the first region. 38. The method of any one of embodiments 32 to 37, wherein the unhybridized region comprises a polyT stretch of at least 5 bases. 39. The method of any one of embodiments 32 to 38, wherein the unhybridized region comprises a plurality of cleavable or excisable bases and wherein cleavage or excision of the plurality of cleavable or excisable bases removes at least a portion of the unhybridized region. 40. The method of embodiment 39, wherein a first cleavable or excisable base is adjacent to a 3′ end of the first region and wherein a second cleavable or excisable base is adjacent to 5′ end of the second region. 41. The method of any one of embodiments 6 to 40, wherein each coupled solid support is coupled to nucleic acid molecules at a density that is less than a predetermined percentage of a maximal density. 42. The method of embodiment 41, wherein the predetermined percentage is about 50%, about 60%, about 70%, about 80%, or about 90%. 43. The method of embodiment 41, wherein the maximal density comprises all first coupling moieties of the coupled solid support coupled to a nucleic acid molecule. 44. The method of any one of embodiments 41 to 43, wherein the density that each coupled solid support is coupled to nucleic acid molecules is less than about 75,000 nucleic acid molecules per cubic micron. 45. The method of embodiment 44, wherein the density that each coupled solid support is coupled to nucleic acid molecules is less than about 20,000 nucleic acid molecules per cubic micron. 46. The method of any one of embodiments 41 to 45, wherein less than 50% of the maximal density comprises at least half of all first coupling moieties not coupled to a nucleic acid molecule. 47. The method of any one of embodiments 1 to 46, wherein the coupled solid support comprises a first portion of the first coupling moieties coupled to the second coupling moiety and a second portion of the first coupling moieties not coupled to the second coupling moiety, and wherein an average distance between adjacent first coupling moieties coupled to the second coupling moiety is at least 2 nm. 48. The method of any one of embodiments 12 to 47, further comprising: (d) contacting the coupled solid supports with a target nucleic acid molecule, wherein the target nucleic acid molecule comprises a 3′ region of reverse complementarity to the single-stranded primers, under conditions sufficient to hybridize the target nucleic acid molecule to the single-stranded primer to produce a solid support coupled to the target nucleic acid molecule. 49. The method of embodiment 48, further comprising: € extending the single-stranded primer from a 3′ end to produce a nucleic acid strand coupled to the solid support and reverse complementary to the target nucleic acid molecule. 50. The method of embodiment 49, further comprising: (f) clonally amplifying the target nucleic acid molecule using the single-stranded primers coupled to the solid support. 51. The method of embodiment 49 or 50, wherein the extending (e), the clonally amplifying (f), or both comprise contacting the solid support coupled to the target nucleic acid molecule with a polymerase and a plurality of free nucleotides.

52. A capture substrate comprising a plurality of solid supports each coupled to a plurality of nucleic acid primers produced by a method of any one of embodiments 1 to 47.

53. A capture substrate, comprising a solid support coupled to a plurality of primers, wherein each primer is hybridized to a complementary nucleic acid molecule and wherein the complementary nucleic acid molecule is devoid of a 5′ unhybridized region of greater than 5 nucleotides. 54. The capture substrate of embodiment 53, wherein the complementary nucleic acid molecule is devoid of a capture entity.

55. A capture substrate, comprising a solid support comprising first coupling moieties, wherein a first portion of the first coupling moieties are coupled to a nucleic acid molecule and a second portion of the first coupling moieties are not coupled to a nucleic acid molecule, and wherein the nucleic acid molecules are present at a density that is less than a predetermined percentage of a maximal density. 56. The capture substrate of embodiment 55, wherein the predetermined percentage is about 50%, about 60%, about 70%, about 80%, or about 90%. 57. The capture substrate of embodiment 56, wherein less than about 50% of the maximal density comprises at least half of all first coupling moieties not coupled to a nucleic acid molecule. 58. The capture substrate of embodiment 55 or 56, wherein at least 10% of the first coupling moieties are coupled to the nucleic acid molecule. 59. The capture substrate of any one of embodiments 55 to 58, wherein the coupled solid support is coupled to nucleic acid molecules at a density of less than about 75,000 nucleic acid molecules per cubic micron. 60. The capture substrate of embodiment 59, wherein the coupled solid support is coupled to nucleic acid molecules at a density of less than about 10,000 nucleic acid molecules per cubic micron. 61. A capture substrate, comprising a solid support comprising between about 5,000 and about 75,000 nucleic acid molecules coupled thereto, wherein the nucleic acid molecules are between 15 and 45 nucleotides in length. 62. The capture substrate of embodiment 61, wherein the solid support comprises between about 5,000 and about 10,000 nucleic acid molecules coupled thereto. 63. The capture substrate of embodiment 61 or 62, wherein the solid support has a diameter of between about 0.2 microns and about 2.5 microns.

64. A capture substrate comprising a solid support comprising a first coupling moiety and an adjacent first coupling moiety, wherein the first coupling moiety and the adjacent first coupling moiety are each coupled to a nucleic acid molecule, and wherein a distance between the first coupling moiety and the adjacent first coupling moiety is at least 2 nm. 65. The capture substrate of embodiment 64, wherein the solid support comprises a plurality of first coupling moieties not coupled to a nucleic acid molecule and wherein an average distance between all adjacent first coupling moieties is less than 2 nm. 66. The capture substrate of embodiment 65, wherein the average distance between all adjacent first coupling moieties is less than 1 nm. 67. The capture substrate of any one of embodiments 53 to 66, wherein the solid support is a bead. 68. The capture substrate of embodiment 67, wherein the bead is a microbead. 69. The capture substrate of embodiment 68, wherein the microbead has a diameter of between about 0.2 microns and about 2.5 microns. 70. The capture substrate of any one of embodiments 55-69, wherein the nucleic acid molecule is a single-stranded primer. 71. The capture substrate of embodiment 70, wherein the single-stranded primer comprises about 15 to about 45 nucleotides. 72. The capture substrate of any one of embodiments 55 to 69, wherein the nucleic acid molecule is a double-stranded nucleic acid molecule and where a strand not coupled to the solid support is devoid of a 5′ single-stranded region of greater than 5 nucleotides. 73. The capture substrate of any one of embodiments 53 to 72, wherein the solid support is coupled to the nucleic acid molecule by a covalent bond. 74. The capture substrate of embodiment 72, wherein the solid support is coupled to the nucleic acid molecule via a click reaction. 75. The capture substrate of any one of embodiments 55 to 74 wherein the solid support is coupled to a 5′ end of the nucleic acid molecule.

76. A composition comprising the capture substrate of any one of embodiments 52 to 75. 77. The composition of embodiment 76, wherein the capture substrate is present at a density sufficient for hybridization between single-stranded nucleic acid molecules of different capture substrates and wherein the capture substrate is a capture substrate of embodiment 53 or 54 or wherein the nucleic acid molecule is a double-stranded nucleic acid molecule.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/210,527, filed Jun. 15, 2021, are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-77. (canceled)

78. A method of controlling density of nucleic acid molecules coupled to solid supports, the method comprising:
a. providing a solid support comprising a plurality of first coupling moieties; and
b. contacting the solid support with a plurality of nucleic acid molecules comprising second coupling moieties under conditions sufficient to couple a first coupling moiety of the plurality of first coupling moieties to a second coupling moiety to produce a coupled solid support, wherein each nucleic acid molecule comprises either:
i. a respective first strand and a respective second strand, wherein each first strand comprises a 5′ linked second coupling moiety configured to couple to the first coupling moiety, or
ii. a third strand comprising a double-stranded region in which a first region of the third strand is hybridized to a second region of the third strand, and wherein the third strand comprises a 5′ linked second coupling moiety configured to couple to the first coupling moiety;
thereby providing a coupled solid support coupled to nucleic acid molecules from the plurality of nucleic acid molecules at a density that is less than a predetermined percentage of a maximal density.

79. The method of claim 78, wherein the maximal density comprises all of the plurality of first coupling moieties of the solid support.

80. The method of claim 78, wherein the predetermined percentage is about 50%, about 60%, about 70%, about 80%, or about 90%.

81. The method of claim 78, wherein the density of the coupled solid support coupled to nucleic acid molecules is less than 2,500 nucleic acid molecules per square micron.

82. The method of claim 78, wherein, for each nucleic acid molecule, at least a portion of the respective first strand is hybridized to at least a portion of the respective second strand.

83. The method of claim 78, wherein the first coupling moiety does not comprise a nucleic acid.

84. The method of claim 78, wherein the first coupling moiety and the second coupling moiety couple by forming a covalent bond.

85. The method of claim 84, wherein the first coupling moiety and the second coupling moiety couple by click chemistry.

86. The method of claim 85, wherein the first coupling moiety is an azide molecule and the second coupling moiety is a diarylcyclooctyne moiety, or wherein the first coupling moiety is a diarylcyclooctyne moiety and the second coupling moiety is an azide molecule.

87. The method of claim 78, wherein the first coupling moiety comprises a nucleic acid, and the first coupling moiety and the second coupling moiety couple by forming a non-covalent bond.

88. The method of claim 78, wherein the third strand further comprises an unhybridized region between the first region and the second region and wherein the unhybridized region comprises at least one cleavable or excisable base.

89. The method of claim 88, further comprising subjecting the coupled solid support to conditions sufficient to cleave or excise the at least one cleavable or excisable base to produce two separate strands.

90. The method of claim 88, wherein the at least one cleavable or excisable base is selected from the group consisting of: a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, a photocleavable base, and any combination thereof.

91. The method of claim 88, wherein the at least one cleavable or excisable base is adjacent to a 3′ end of the first region.

92. The method of claim 88, wherein the unhybridized region comprises a polyT stretch of at least 5 bases.

93. The method of claim 88, wherein a first cleavable or excisable base is adjacent to a 3′ end of the first region and wherein a second cleavable or excisable base is adjacent to 5′ end of the second region.

94. The method of claim 78, wherein a nucleic acid molecule in the plurality of nucleic acid molecules coupled to the solid support comprises a primer hybridized thereto.

95. The method of claim 94, further comprising:
e. extending the primer from a 3′ end to produce a nucleic acid strand that is coupled to the solid support and has reverse complementary to a target nucleic acid molecule.

96. The method of claim 95, further comprising:
f. clonally amplifying the target nucleic acid molecule using primers coupled to the solid support.

97. The method of claim 78, wherein the solid support is a bead.
